(19) United States
(12) Patent Application Publication
(10) Pub. No.: US 2020/0315967 A1
(43) Pub. Date: Oct. 8, 2020

(54) LIPID NANOPARTICLES

(71) Applicant: ModernaTX, Inc., Cambridge, MA (US)

(72) Inventor: Benjamin Frank GELDHOF, Winthrop, MA (US)

(73) Assignees: ModernaTX, Inc., Cambridge, MA (US); ModernaTX, Inc., Cambridge, MA (US)

(21) Appl. No.: 16/304,043

(22) PCT Filed: Jun. 21, 2017
(86) PCT No.: PCT/US2017/038426

§ 371 Date: Nov. 21, 2018

Related U.S. Application Data
(60) Provisional application No. 62/354,351, filed on Jun. 24, 2016.

(51) Int. Cl. A61K 9/127; A61K 9/14; A61M 5/20; A61K 9/08; A61K 31/7105

(52) U.S. Cl. CPC A61K 9/1271; A61K 9/145; A61M 5/20; B82Y 5/00; A61K 31/7105; A61K 9/1277; A61K 9/08

(57) ABSTRACT

The invention features methods and apparatus for producing lipid nanoparticles. Methods of the invention include injecting a lipid solution into an aqueous solution at an automated rate (e.g., a rate controlled by a servo pump). The invention provides methods and apparatus for making lipid nanoparticles possessing a wide range of lipid components and hydrophilic encapsulants, including nucleic acids (e.g., mRNA). Also provided are nanoparticles and compositions thereof made by methods and apparatus of the invention.

### BACKGROUND OF THE INVENTION

Nanoparticles are useful for the delivery of various therapeutic, diagnostic, or experimental agents to cells and tissues. Nanoparticles are hypothesized to have enhanced interfacial cellular uptake because of their sub-cellular size, achieving a local effect. It is also hypothesized that there is enhanced cellular uptake of agents encapsulated in nanoparticles compared to the corresponding agent administered in free form. Thus, nanoparticle-entrapped agents have enhanced and sustained concentrations inside cells, thereby increasing therapeutic effects. Furthermore, nanoparticle-entrapped agents are protected from metabolic inactivation before reaching the target site, as often happens upon systemic administration of free agents. Therefore, the effective local nanoparticle dose required for the local pharmacologic effect may be several fold lower than with systemic or oral doses. Lipid nanoparticles, in particular, are useful in enhancing the delivery of agents such as nucleic acids.

Widespread utility of lipid nanoparticles is limited, in part, due to manufacturing and processing constraints. In particular, large-scale production of lipid nanoparticle formulations can introduce variability in lipid nanoparticle characteristics, such as chemical composition, surface charge, size, batch-to-batch concentration, and purity. Such processing limitations have generated a need in the field for new methods and apparatus for synthesizing lipid nanoparticles.

### SUMMARY OF THE INVENTION

The invention provides a method for producing lipid nanoparticles, the method including the steps of providing an aqueous solution; providing a lower alkanol solution including lipids; and injecting at an automated rate (e.g., at a rate controlled by a servo pump) the lower alkanol solution to the aqueous solution to produce the lipid nanoparticles. In some embodiments, the steps of the method of the invention are repeated one or more (e.g., 1, 2, 3, 4, 5, 6, 7, 8, 9, 10, or more) times.

In some embodiments, the lipid nanoparticles have a mean diameter between 80 nm and 100 nm (e.g., between 82 nm and 98 nm, between 84 nm and 96 nm, between 86 nm and 94 nm, between 88 nm and 92 nm, about 80 nm, about 81 nm, about 82 nm, about 83 nm, about 84 nm, about 85 nm, about 86 nm, about 87 nm, about 88 nm, about 89 nm, about 90 nm, about 91 nm, about 92 nm, about 93 nm, about 94 nm, about 95 nm, about 96 nm, about 97 nm, about 98 nm, about 99 nm, or about 100 nm) and a polydispersity index of 0.25 or less (e.g., 0.25, 0.24, 0.23, 0.22, 0.21, 0.20, 0.19, 0.18, 0.17, 0.16, 0.15, 0.14, 0.13, 0.12, 0.11, 0.10, 0.09, 0.08, 0.07, 0.06, 0.05, 0.04, 0.03, 0.02, 0.01, or less).

In some embodiments, the aqueous solution includes a nucleic acid (e.g., DNA, RNA, e.g., mRNA). The nucleic acid may be at a concentration between 50 μg per ml and 200 μg per ml of the aqueous solution (e.g., about 50 μg/ml, about 60 μg/ml, about 70 μg/ml, about 80 μg/ml, about 90 μg/ml, about 100 μg/ml, about 110 μg/ml, about 111 μg/ml, about 111.11 μg/ml, about 120 μg/ml, about 130 μg/ml, about 140 μg/ml, about 150 μg/ml, about 175 μg/ml, or about 200 μg/ml). All of or a portion of the nucleic acid may be encapsulated in the lipid nanoparticles. In some embodiments, the method yields a nucleic acid encapsulation efficiency of at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or greater). In some embodiments, the method yields a nucleic acid encapsulation efficiency of at least 94%.

In some embodiments, the lower alkanol solution provides 50% or less (e.g., between 25% and 50%, e.g., 50%, 49%, 48%, 47%, 46%, 45%, 44%, 43%, 42%, 41%, 40%, 39%, 38%, 37%, 36%, 35%, 34%, 33%, 32%, 31%, 30%, 29%, 28%, 27%, 26%, 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2%, 1%, or less) of the total volume. In some embodiments, the lower alkanol solution provides about 33% or about 25% of the total volume.

In some embodiments, the injecting is at a rate from about 1,000 to about 5,000 microliters per second (μl/s; e.g., from about 1,000 to about 5,000 μl/s, from about 1,500 to about 4,500 μl/s, from about 2,000 to about 4,000 μl/s, or from about 2,500 to about 3,000 μl/s). For example, injecting may be at a rate of 2,600 μl/s.

In some embodiments, the aqueous solution further includes a buffer. Examples of suitable buffers include, but are not limited to, a citrate buffer (e.g., 100 mM citrate buffer), a phosphate buffer (e.g., phosphate buffered saline (PBS)), or a TRIS buffer (e.g., TRIS/Sucrose). In some embodiments, the aqueous solution has a pH from about 3.0 to about 8.0 (e.g., about 3.0, about 3.1, about 3.2, about 3.3, about 3.4, about 3.5, about 3.6, about 3.7, about 3.8, about 3.9, about 4.0, about 4.1, about 4.2, about 4.3, about 4.4, about 4.5, about 4.6, about 4.7, about 4.8, about 4.9, about 5.0, about 5.1, about 5.2, about 5.3, about 5.4, about 5.5, about 5.6, about 5.7, about 5.8, about 5.9, about 6.0, about 6.1, about 6.2, about 6.3, about 6.4, about 6.5, about 6.6, about 6.7, about 6.8, about 6.9, about 7.0, about 7.1, about 7.2, about 7.3, about 7.4, about 7.5, about 7.6, about 7.7, about 7.9, or about 8.0). The aqueous solution can have an osmolality from about 200 mOsm to about 400 mOsm (e.g., from about 250 mOsm to about 350 mOsm, or about 260 mOsm, 270 mOsm, 280 mOsm, 290 mOsm, 300 mOsm, 310 mOsm, 320 mOsm, 330 mOsm, 340 mOsm, or 350 mOsm).

In some embodiments, the lower alkanol solution includes heptatriaconta-6,9,28,31-tetraen-19-yl-4-(dimethylamino)butanoate (DLin-MC3-DMA), phosphatidylcholine (1,2-distearoyl-sn-glycero-3-phosphocholine (DSPC), cholesterol, and polyethylene glycol-dimyristolglycerol (PEG-DMG). The ratio of DLin-MC3-DMA:DSPC:Cholesterol:PEG-DMG may be, for example, 50:10:38.5:1.5.

In some embodiments, the method further includes purifying and/or concentrating the dispersion of lipid nanoparticles, e.g., through the use of a desalting column, dialysis, or tangential flow filtration. Purification and/or concentration may be performed as part of a buffer exchange procedure, e.g., complete buffer exchange. The method may further include sterilizing the dispersion of lipid nanoparticles by filtration, e.g., microfiltration.

In some embodiments, the invention provides a method of producing nanoparticles having an encapsulation efficiency of greater than 90% (e.g., 90%, 91%, 92%, 93%, 94%, 95%, 96%, 97%, 98%, 99%, or 100%).

In another aspect, the invention provides a lipid nanoparticle produced by injecting a lower alkanol solution into an aqueous solution having a lipid, wherein the injecting is automated at a rate from about 1,000 to about 5,000 microliters per second (μl/s; e.g., from about 1,000 to about 5,000 μl/s, from about 1,500 to about 4,500 μl/s, from about 2,000 to about 4,000 μl/s, or from about 2,500 to about 3,000 μl/s). In some embodiments, the injecting is at a rate of 2,600 μl/s.

In another aspect, the invention provides an apparatus for producing lipid nanoparticles, the apparatus having an injector configured to transfer a lower alkanol solution from a first reservoir to a second reservoir configured to hold an aqueous solution; and a servo pump configured to operate the injector at a rate from 1,000 to 5,000 μl/s (e.g., from 1,500 to 4,000 μl/s, 2,000 to 3,500 μl/s, or from 2,500 to 3,000 μl/s. In some embodiments, the servo pump is configured to operate the injector at a rate of 2,600 μl/s.

In some embodiments, the injector is configured to move in three dimensions relative to the second reservoir. In some embodiments, the injector is configured to move in three dimensions relative to the first reservoir and second reservoir.

In some embodiments, the invention provides a pharmaceutical composition including the lipid nanoparticles and a pharmaceutically acceptable carrier.

### BRIEF DESCRIPTION OF THE DRAWINGS

FIG. 1 is a schematic diagram of an injector including a stepper motor, lead screw with plunger, syringe barrel, pipette tip adaptor, and pipette tip.

### DEFINITIONS

The term “nucleic acid” refers to a molecule of two or more nucleotides or alternative nucleotides. The term, “nucleotide” refers to a nucleoside including a phosphate group. The term “nucleoside” refers to a compound containing a sugar molecule (e.g., a pentose or ribose) or derivative thereof in combination with an organic base (e.g., a purine or pyrimidine) or a derivative thereof (also referred to herein as a “nucleobase”). Examples of nucleic acids include but are not limited to DNA, RNA, tRNA (transfer RNA), mRNA (messenger RNA), siRNA (small interfering RNA), miRNA (micro RNA), shRNA (short hairpin RNA), ncRNA (non-coding RNA), aptamers, ribozymes, and shorter oligonucleotide sequences of any of the foregoing. Alterations of the base, sugar, and phosphate moiety of a nucleotide are encompassed by this definition. Herein, in a nucleotide, nucleoside or polynucleotide (such as the nucleic acids of the invention, e.g., mRNA molecule), the terms “alteration” or, as appropriate, “alternative” refer to alteration with respect to A, G, U or C ribonucleotides. Generally, herein, these terms are not intended to refer to the ribonucleotide alterations in naturally occurring 5′-terminal mRNA cap moieties.

As used herein, the terms “alteration” or “alternative” of a nucleotide, nucleoside, or polynucleotide (such as the polynucleotides of the invention, e.g., mRNA molecule), refer to alteration with respect to A, G, U or C ribonucleotides. Generally, herein, these terms are not intended to refer to the ribonucleotide alterations in naturally occurring 5′-terminal mRNA cap moieties.

As used herein, the term “nanoparticle” refers to a particle having one or a plurality of components, the particle having any one structural feature on a scale of less than about 1000 nm that exhibits novel properties as compared to a bulk sample of the same material or component materials. Routinely, nanoparticles have any one structural feature on a scale of less than about 500 nm, less than about 400 nm, less than about 300 nm, less than about 200 nm or less than about 100 nm. In exemplary embodiments, a nanoparticle is a particle having one or more dimensions of the order of about 10-500 nm. In other exemplary embodiments, a nanoparticle is a particle having one or more dimensions of the order of about 10-1000 nm. A spherical nanoparticle would have a diameter, for example, of between 10-100 nm or 10-1000 nm.

A nanoparticle most often behaves as a unit in terms of its physical or biophysical properties, e.g., transport. It is noted that novel properties that differentiate nanoparticles from the corresponding bulk material typically develop at a size scale of under 1000 nm, or at a size of under 500 nm, but nanoparticles can be of a larger size, for example, for particles that are oblong, tubular, and the like. The size at which materials display different properties as compared to the bulk material is material-dependent and can be seen for many materials much larger in size than 100 nm and even for some materials larger in size than 1000 nm. Nanoparticles can be employed in a variety of drug delivery technologies (e.g., nucleic acid drug delivery technologies) and can be employed for various purposes including, but not limited to, controlled drug delivery, protection of the drugs from degradation, and protection of the body from the toxic effects of the drugs.

As used herein, the term “microparticle” refers to a small particle or particulate system, generally larger than about one micrometer (1 μm) in diameter and can be used to describe both microcapsules and microspheres.

The term “lipid nanoparticle” refers to a nanoparticle as described above that includes lipids and that is stable and dispersible in aqueous media. In exemplary embodiments, lipid nanoparticles may be from 10 nm to 500 nm in diameter, e.g., from 70 nm to 120 nm.

The term “lower alkanol” refers to an alcohol with 6 or fewer carbon atoms. Examples of lower alkanols include but are not limited to methanol, ethanol, propanol, pentanol, their isomers, and mixtures thereof.

The term “injector” refers to a structure, through which a solution passes, that is configured to guide the flow of a solution into a reservoir.

The term “injection rate” refers to the volume of fluid that is injected per unit time.

The term “total volume after injecting” refers to the volume of suspension (e.g., including the lower alkanol solution, the aqueous solution, and any encapsulants) at a time point immediately following the termination of injecting, prior to any subsequent processing, such as filtration, lyophilization, etc.

The term “particle size” or “particle diameter” refers to the mean diameter of the particles in a sample, as measured by dynamic light scattering (DLS), multiangle light scattering (MALS), nanoparticle tracking analysis, or comparable techniques. It will be understood that a dispersion of lipid nanoparticles as described herein will not be of uniform size but can be described by the average diameter and, optionally, the polydispersity index.

In preferred embodiments, the lipid nanoparticles in the formulation of the present invention have a single mode particle size distribution (i.e., they are not bi- or poly-modal). The particle size distribution relates to the amount of particles by size within a given population. This is derived using Mie theory, where the assumption is that all particles are spherical, and the optical properties of the particles are known. A particle size distribution can be measured by dynamic light scattering (DLS) or other particle tracking systems (e.g., diffraction tracking and Brownian motion analysis).

The term “encapsulation efficiency” as used herein refers to the percentage of nucleic acid in the lipid nanoparticles that is not degraded after exposure to serum or a nuclease assay that would significantly degrade free nucleic acids. Encapsulation efficiency can be measured as follows: Dilute the lipid nanoparticle formulation to about 1-10 μg/mL in 1×TE buffer. Place 50 μl of the sample in a well in a polystyrene 96 well plate, and 50 μl in the well below it. Add 50 μl of 1×TE buffer to the top well, and 50 of 2% Triton X-100 to the bottom well. For the reference wells, replace the sample with 50 μl of 1×TE buffer. Allow the 96 well plate to incubate at 37° C. for 15 minutes. During this time, allow RiboGreen® to thaw. Once thawed, dilute the RiboGreen 1:100 in 1×TE buffer. After the 15 minute incubation, add 100 μl of diluted RiboGreen reagent to each well, mixing thoroughly by pipetting. Once addition of the RiboGreen is complete, the plate is then read by a fluorescence plate reader (FITC settings); after subtracting the fluorescence values of the blanks from each sample well, the percent of free mRNA may be determined by dividing the fluorescence of the intact liposome sample (no Triton X-100) by the fluorescence value of the disrupted liposome sample (with Triton X-100).

Entrapped fraction=1−free fraction.

Encapsulation efficiency=100×entrapped fraction.

The term “fully encapsulated” as used herein indicates that the nucleic acid in the particles is not significantly degraded after exposure to serum or a nuclease assay that would significantly degrade free nucleic acids. In a fully encapsulated system, preferably less than 25% of particle nucleic acid is degraded in a treatment that would normally degrade 100% of free nucleic acid, more preferably less than 10% and most preferably less than 5% of the particle nucleic acid is degraded. Fully encapsulated also indicates that the particles are serum stable, that is, that they do not rapidly decompose into their component parts upon in vivo administration.

### DETAILED DESCRIPTION

The invention provides methods and apparatus for producing lipid nanoparticles that involve injecting a lipid solution into an aqueous solution at an automated rate. The methods can be used to make lipid nanoparticles possessing a wide range of lipid components including, but not limited to, cationic lipids, anionic lipids, neutral lipids, polyethylene glycol (PEG) lipids, hydrophilic polymer lipids, fusogenic lipids, and sterols. Hydrophobic agents can be incorporated into the organic solvent (e.g., ethanol) with the lipid, and nucleic acids can be added to an aqueous component. The methods and apparatus can be used to prepare homogeneous dispersions of lipid nanoparticles.

### Methods

Lipid nanoparticles can be produced (e.g., allowed to self-assemble, e.g., spontaneously) by injecting a lower alkanol solution containing lipids into an aqueous solution. Various lipids can be used to achieve desired properties, such as size, surface charge, and capacity for encapsulants. Such properties can also be influenced by the composition of the aqueous solution. Lipid nanoparticles of the invention can encapsulate a wide range of hydrophilic molecules, e.g., nucleic acids such as DNA and RNA, or alternative versions of DNA or RNA. Variations in lipid nanoparticle size can be affected by controlling process parameters. In particular, the rate at which the lower alkanol solution is injected into the aqueous solution is inversely related to the resulting lipid nanoparticle size. Similarly, minimizing variance in the rate of injection will minimize variance in lipid nanoparticle size, yielding homogeneous suspensions of lipid nanoparticles, e.g., within a single batch or among multiple batches. A precise rate of injection can be attained, e.g., through a servo pump.

Typically, the lipid nanoparticles are liposomes with a lipid bilayer surrounding an aqueous interior. Liposomes can be of different sizes such as, but not limited to, a multilamellar vesicle (MLV) which may be hundreds of nanometers in diameter and may contain a series of concentric bilayers separated by narrow aqueous compartments, a small unicellular vesicle (SUV) which may be smaller than 50 nm in diameter, and a large unilamellar vesicle (LUV) which may be between 50 nm and 500 nm in diameter. Liposome design may include, but is not limited to, opsonins or ligands in order to improve the attachment of liposomes to unhealthy tissue or to activate events such as, but not limited to, endocytosis. Liposomes may contain a low or a high pH in order to improve the delivery of the pharmaceutical formulations.

The formation of liposomes may depend on the physicochemical characteristics such as, but not limited to, the pharmaceutical formulation entrapped and the liposomal ingredients, the nature of the medium in which the lipid vesicles are dispersed (e.g., osmolality or pH), the effective concentration of the entrapped substance and its potential toxicity, any additional processes involved during the application and/or delivery of the vesicles, the optimization size, polydispersity and the shelf-life of the vesicles for the intended application, and the batch-to-batch reproducibility and possibility of large-scale production of safe and efficient liposomal products.

The invention further provides methods of injecting the lipid-containing lower alkanol solution into the aqueous solution at a precise rate. Without wishing to be bound by theory, a high rate of injection creates greater turbulence and provides more energy for lipid assembly, yielding small lipid nanoparticles. In some embodiments, the volume of the aqueous solution is at most 5.0 ml. In conditions in which the volume of the aqueous solution is 5.0 ml, the rate of injection of the lower alkanol solution can be between 1,000 μl/s and 5,000 μl/s (e.g., between 1,500 μl/s and 4,000 μl/s, between 2,000 and 3,000 μl/s, between 2,400 μl/s and 2,800 μl/s, or about 2,600 μl/s), or between 20% and 100% (e.g., between 30% and 80%, between 40% and 60%, or between 48% and 56%, e.g., about 52%) of the total volume per second. Lipid nanoparticles of the invention may have a diameter of, e.g., between 70 nm and 110 nm (e.g., between 75 nm and 105 nm, between 80 nm and 100 nm, between 82 nm and 98 nm, between 84 nm and 96 nm, between 86 nm and 94 nm, between 88 nm and 92 nm, about 85 nm, about 90 nm, or about 95 nm) with a polydispersity index of 0.25 or less (e.g., 0.25, 0.24, 0.23, 0.22, 0.21, 0.20, 0.19, 0.18, 0.17, 0.16, 0.15, 0.14, 0.13, 0.12, 0.11, 0.10, 0.09, 0.08, 0.07, 0.06, 0.05, 0.04, 0.03, 0.02, 0.01, or less), as measured by dynamic light scattering (DLS). In some embodiments, all or a portion of the nucleic acid is encapsulated in the lipid nanoparticles. In some embodiments, the method yields a nucleic acid encapsulation efficiency of at least 80% (e.g., at least 80%, at least 85%, at least 90%, at least 92%, at least 94%, at least 95%, at least 96%, at least 97%, or at least 98%, e.g., 80%, 81%, 82%, 83%, 84%, 85%, 86%, 87%, 88%, 89%, 90%, 91%, 92%, 93%, 94%, 95%, 96%, 97%, 98%, 99%, or greater).

The volume fraction may be modified according to parameters known in the art. For example, a lower alkanol solution having a greater concentration of lipid molecules may necessitate a lesser volume of lower alkanol solution relative to aqueous solution, e.g., to maintain the molar ratio of lipid to hydrophilic encapsulants.

The lower alkanol solution can be injected automatically (e.g., at an automated rate, e.g., by an automatic injector, e.g., a servo-powered injector, e.g., as part of a robotic pipetting apparatus) into a volume of aqueous solution, e.g., contained within a reservoir, e.g., a well of a multi-well plate, test tube, or flask. Reservoirs can be part of a multi-reservoir unit, e.g., a multi-well plate or a rack of multiple tubes (e.g., 0.25 ml tubes, 0.5 ml tubes, 1.0 ml tubes, 1.5 ml tubes, 2.0 ml tubes, 2.5 ml tubes, 5.0 ml tubes, 10 ml tubes, 15 ml tubes, or 50 ml tubes, e.g., included as part of a multiple tube rack, e.g., a 2-tube rack, a 4-tube rack, a 6-tube rack, an 8-tube rack, a 10-tube rack, a 12-tube rack, a 16 tube rack, a 24-tube rack, a 48 tube rack, or a 96-tube rack). In some embodiments, one or more multi-reservoir units (e.g., tube racks) can be processed in a single run. The aqueous solution may be stationary or under mixing or agitation. Multiple, repeated injections, each into a corresponding reservoir of aqueous solution, can produce multiple suspensions of lipid nanoparticles. The injector can be moved relative to the aqueous solution reservoir, or, alternatively, the aqueous solution reservoir can be moved relative to the injector. An injector may additionally move in the Z direction, relative to the aqueous solution reservoir, e.g., such that ejection of lower alkanol solution from the injector occurs below the surface of the aqueous solution. Alternatively, ejection of the lower alkanol solution from the injector can occur above the surface of the aqueous solution, e.g., to prevent spillage and/or crossover of aqueous solution between reservoirs.

The total volume of the lower alkanol solution per injection is generally equal to or less than the total volume of suspension upon injection completion (e.g., the mixture of lower alkanol solution and aqueous solution). In some embodiments, the volume of the lower alkanol solution per injection is between 10% and 100% (e.g., between 20% and 90%, between 30% and 80%, between 40% and 75%, between 50% and 70%, or between 60% and 70%, e.g., about 20%, about 30%, about 40%, about 50%, about 60%, about 65%, about 66.67%, about 70%, about 80%, about 90%, or about 100%) of the volume of the aqueous solution or between 5% and 50%, between 10% and 45%, between 15% and 40%, between 20% and 38%, or between 25% and 35%, e.g., about 20%, about 30%, about 33%, about 40%, or about 40% of the total volume of suspension upon injection completion). Other volume fractions can be used. In general, the greater the volume of the lower alkanol solution relative to the aqueous solution, the more concentrated the suspension of lipid nanoparticles will be upon completion of injection.

Conventional downstream processing can be employed as part of the present invention. For example, lipid nanoparticles can be purified or concentrated, e.g., by tangential flow filtration, dialysis, or desalting column (e.g., a PD-10 desalting column). In methods involving dialysis, the filter membrane geometry, including the area of the filter and the fiber diameter can be varied to achieve an optimal rate of filtration according to known parameters, such as lipid nanoparticle size, encapsulants size, and liquid viscosity (e.g., buffer viscosity). A person of skill in the art will understand that the effect of varying each of these parameters can be informed by the Stokes-Einstein equation, below, where D is the diffusion constant of a particle, k_{B} is Boltzmann's constant, T is temperature, η is viscosity, and r is the radius of the particle.

$$D = \frac{{k}_{B} T}{6 π η r}$$

For tangential flow filtration procedures, the effect of liquid flow reduces the influence of diffusion on buffer exchange rate. In this case, increasing the recirculation rate will increase the shear rate and enhance the rate of buffer exchange. The transmembrane and permeate pressures can also be varied.

In some embodiments, the nanoparticle suspension can be exchanged with a solution containing a cryoprotectant (e.g., for long term storage in, e.g., frozen or lyophilized form). Physiologically suitable cyroprotectants for lipid nanoparticles are known in the art and include, e.g., sucrose, glucose, mannitol, glycerol, and other carbohydrates and polyalcohols. In one non-limiting example, a lipid nanoparticle solution is dialyzed against a sucrose solution (e.g., a TRIS/sucrose buffer).

A sterile filtration step may also be employed, and the membrane area, pore size and filtration force can be varied, as described above. The lipid nanoparticles described herein may be made in a sterile environment by the system and/or methods described in U.S. Publication No. 20130164400.

### Lower Alkanol Solution

A lower alkanol solution of the invention provides the lipid components that assemble into lipid nanoparticles upon injection into the aqueous solution. Lipid components that can be included in the lower alkanol solution include, but are not limited to, cationic lipids, anionic lipids, neutral lipids, polyethyleneglycol lipids, hydrophilic polymer lipids, and fusogenic lipids.

In one embodiment, the lower alkanol solution includes at least one lipid. The lipid may be selected from, but is not limited to, L604, DLin-DMA, DLin-K-DMA, 98N12-5, C12-200, DLin-MC3-DMA, DLin-KC2-DMA, DODMA, PLGA, PEG, PEG-DMG, PEGylated lipids and amino alcohol lipids. In another aspect, the lipid may be a cationic lipid such as, but not limited to, DLin-DMA, DLin-D-DMA, DLin-MC3-DMA, DLin-KC2-DMA, DODMA and amino alcohol lipids. The amino alcohol cationic lipid may be the lipids described in and/or made by the methods described in U.S. Publication No. US20130150625, herein incorporated by reference in its entirety. As a non-limiting example, the cationic lipid may be 2-amino-3-[(9Z,12Z)-octadeca-9,12-dien-1-yloxy]-2-{[(9Z,2Z)-octadeca-9,12-dien-1-yloxy]methyl}propan-1-ol (Compound 1 in US20130150625); 2-amino-3-[(9Z)-octadec-9-en-1-yloxy]-2-{[(9Z)-octadec-9-en-1-yloxy]methyl}propan-1-ol (Compound 2 in US20130150625); 2-amino-3-[(9Z,12Z)-octadeca-9,12-dien-1-yloxy]-2-[(octyloxy)methyl]propan-1-ol (Compound 3 in US20130150625); and 2-(dimethylamino)-3-[(9Z,12Z)-octadeca-9,12-dien-1-yloxy]-2-{[(9Z,12Z)-octadeca-9,12-dien-1-yloxy]methyl}propan-1-ol (Compound 4 in US20130150625); or any pharmaceutically acceptable salt or stereoisomer thereof.

Lipid nanoparticle formulations may include, e.g., an ionizable cationic lipid, for example, 2,2-dilinoleyl-4-dimethylaminoethyl-[1,3]-dioxolane (DLin-KC2-DMA), dilinoleyl-methyl-4-dimethylaminobutyrate (DLin-MC3-DMA), or di((Z)-non-2-en-1-yl) 9-((4-(dimethylamino)butanoyl)oxy)heptadecanedioate (L319), and further comprise a neutral lipid, a sterol and a molecule capable of reducing particle aggregation, for example a PEG or PEG-modified lipid.

In one embodiment, the lower alkanol solution includes (i) at least one lipid selected from the group consisting of 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319); (ii) a neutral lipid selected from DSPC, DPPC, POPC, DOPE and SM; (iii) a sterol, e.g., cholesterol; and (iv) a PEG-lipid, e.g., PEG-DMG or PEG-cDMA, in a molar ratio of about 20-60% cationic lipid: 5-25% neutral lipid: 25-55% sterol; 0.5-15% PEG-lipid.

In one embodiment, the lower alkanol solution includes from about 25% to about 75% on a molar basis of a cationic lipid selected from 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319), e.g., from about 35 to about 65%, from about 45 to about 65%, about 60%, about 57.5%, about 50% or about 40% on a molar basis.

In one embodiment, the lower alkanol solution includes from about 0.5% to about 15% on a molar basis of the neutral lipid e.g., from about 3 to about 12%, from about 5 to about 10% or about 15%, about 10%, or about 7.5% on a molar basis. Exemplary neutral lipids include, but are not limited to, DSPC, POPC, DPPC, DOPE and SM. In one embodiment, the lower alkanol solution includes from about 5% to about 50% on a molar basis of the sterol (e.g., about 15 to about 45%, about 20 to about 40%, about 40%, about 38.5%, about 35%, or about 31% on a molar basis. An exemplary sterol is cholesterol. In one embodiment, the lower alkanol solution includes from about 0.5% to about 20% on a molar basis of the PEG or PEG-modified lipid (e.g., about 0.5 to about 10%, about 0.5 to about 5%, about 1.5%, about 0.5%, about 1.5%, about 3.5%, or about 5% on a molar basis). In one embodiment, the PEG or PEG modified lipid comprises a PEG molecule of an average molecular weight of 2,000 Da. In other embodiments, the PEG or PEG modified lipid comprises a PEG molecule of an average molecular weight of less than 2,000, for example around 1,500 Da, around 1,000 Da, or around 500 Da. Exemplary PEG-modified lipids include, but are not limited to, PEG-distearoyl glycerol (PEG-DMG) (also referred herein as PEG-C14 or C14-PEG), PEG-cDMA (see, e.g., Reyes et al. J. Controlled Release 2005, 107, 276-287).

In one embodiment, the lower alkanol solution includes 25-75% (e.g., 35%-65%, 45-65%, about 60%, about 50%, about 40%, or about 57.5%) of a cationic lipid selected from 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319), 0.5-15% of the neutral lipid, 5-50% of the sterol, and 0.5-20% of the PEG or PEG-modified lipid on a molar basis.

In some embodiments, the lower alkanol solution includes a lipid mixture in molar ratios of about 20-70% cationic lipid: 5-45% neutral lipid: 20-55% cholesterol: 0.5-15% PEG-modified lipid. In some embodiments, the lower alkanol solution includes a lipid mixture in molar ratios of about 20-60% cationic lipid: 5-25% neutral lipid: 25-55% cholesterol: 0.5-15% PEG-modified lipid.

In particular embodiments, the molar lipid ratio is approximately 50/10/38.5/1.5 (mol % cationic lipid/neutral lipid, e.g., DSPC/Chol/PEG-modified lipid, e.g., PEG-DMG, PEG-DSG or PEG-DPG), 57.2/7.1134.3/1.4 (mol % cationic lipid/neutral lipid, e.g., DPPC/Chol/PEG-modified lipid, e.g., PEG-cDMA), 40/15/40/5 (mol % cationic lipid/neutral lipid, e.g., DSPC/Chol/PEG-modified lipid, e.g., PEG-DMG), 50/10/35/4.5/0.5 (mol % cationic lipid/neutral lipid, e.g., DSPC/Chol/PEG-modified lipid, e.g., PEG-DSG), 50/10/35/5 (cationic lipid/neutral lipid, e.g., DSPC/Chol/PEG-modified lipid, e.g., PEG-DMG), 40/10/40/10 (mol % cationic lipid/neutral lipid, e.g., DSPC/Chol/PEG-modified lipid, e.g., PEG-DMG or PEG-cDMA), 35/15/40/10 (mol % cationic lipid/neutral lipid, e.g., DSPC/Chol/PEG-modified lipid, e.g., PEG-DMG or PEG-cDMA), or 52/13/30/5 (mol % cationic lipid/neutral lipid, e.g., DSPC/Chol/PEG-modified lipid, e.g., PEG-DMG or PEG-cDMA).

Exemplary lipid nanoparticle compositions and methods of making same are described, for example, in Semple et al. (Nat. Biotechnol. 2010, 28:172-176), Jayarama et al. (Angew. Chem. Int. Ed. 2012, 51: 8529-8533), and Maier et al. (Molecular Therapy 2013, 21, 1570-1578).

In one embodiment, the lower alkanol solution may include a cationic lipid, a PEG lipid and a structural lipid and optionally comprise a non-cationic lipid. As a non-limiting example, the lower alkanol solution may include about 40-60% of cationic lipid, about 5-15% of a non-cationic lipid, about 1-2% of a PEG lipid and about 30-50% of a structural lipid. As another non-limiting example, the lower alkanol solution may include about 50% cationic lipid, about 10% non-cationic lipid, about 1.5% PEG lipid and about 38.5% structural lipid. As yet another non-limiting example, the lower alkanol solution may include about 55% cationic lipid, about 10% non-cationic lipid, about 2.5% PEG lipid, and about 32.5% structural lipid. In one embodiment, the cationic lipid may be any cationic lipid described herein such as, but not limited to, DLin-KC2-DMA, DLin-MC3-DMA, and L319.

In one embodiment, the lower alkanol solutions described herein may include four components. The lower alkanol solution may include a cationic lipid, a non-cationic lipid, a PEG lipid and a structural lipid. As a non-limiting example, the lower alkanol solution may include about 40-60% of cationic lipid, about 5-15% of a non-cationic lipid, about 1-2% of a PEG lipid and about 30-50% of a structural lipid. As another non-limiting example, the lower alkanol solution may include about 50% cationic lipid, about 10% non-cationic lipid, about 1.5% PEG lipid and about 38.5% structural lipid. As yet another non-limiting example, the lower alkanol solution may include about 55% cationic lipid, about 10% non-cationic lipid, about 2.5% PEG lipid, and about 32.5% structural lipid. In one embodiment, the cationic lipid may be any cationic lipid described herein such as, but not limited to, DLin-KC2-DMA, DLin-MC3-DMA and L319.

In one embodiment, the lower alkanol solution described herein may comprise a cationic lipid, a non-cationic lipid, a PEG lipid and a structural lipid. As a non-limiting example, the lower alkanol solution may include about 50% of the cationic lipid DLin-KC2-DMA, about 10% of the non-cationic lipid DSPC, about 1.5% of the PEG lipid PEG-DOMG and about 38.5% of the structural lipid cholesterol. As a non-limiting example, the lower alkanol solution may include about 50% of the cationic lipid DLin-MC3-DMA, about 10% of the non-cationic lipid DSPC, about 1.5% of the PEG lipid PEG-DOMG and about 38.5% of the structural lipid cholesterol. As a non-limiting example, the lower alkanol solution may include about 50% of the cationic lipid DLin-MC3-DMA, about 10% of the non-cationic lipid DSPC, about 1.5% of the PEG lipid PEG-DMG and about 38.5% of the structural lipid cholesterol. As yet another non-limiting example, the lower alkanol solution may include about 55% of the cationic lipid L319, about 10% of the non-cationic lipid DSPC, about 2.5% of the PEG lipid PEG-DMG and about 32.5% of the structural lipid cholesterol.

Other cationic lipids include, but are not limited to, a cationic lipid described in International Patent Publication Nos. WO2012040184, WO2011153120, WO2011149733, WO2011090965, WO2011043913, WO2011022460, WO2012061259, WO2012054365, WO2012044638, WO2010080724, WO201021865, WO2008103276, WO2013086373, and WO2013086354, U.S. Pat. Nos. 7,893,302, 7,404,969, 8,283,333, 8,466,122, and 8,569,256, and U.S. Publication No. 20100036115, 20120202871, 20130064894, 20130129785, 20130150625, 20130178541, 20130225836, and 20140039032. In another embodiment, the cationic lipid may be selected from, but not limited to, formula A described in International Patent Publication Nos. WO2012040184, WO2011153120, WO2011149733, WO2011090965, WO2011043913, WO2011022460, WO2012061259, WO2012054365, WO2012044638, and WO2013116126, or U.S. Publication Nos. 20130178541 and 20130225836. In yet another embodiment, the cationic lipid may be selected from, but not limited to, formula CLI-CLXXIX of International Patent Publication No. WO2008103276, formula CLI-CLXXIX of U.S. Pat. No. 7,893,302, formula CLI-CLXXXXII of U.S. Pat. No. 7,404,969, formula I-VI of U.S. Publication No. 20100036115, and formula I of U.S. Publication No 20130123338. As a non-limiting example, the cationic lipid may be selected from (20Z,23Z)-N,N-dimethylnonacosa-20,23-dien-10-amine, (17Z,20Z)-N,N-dimemylhexacosa-17,20-dien-9-amine, (1Z,19Z)-N,N-dimethylpentacosa-16,19-dien-8-amine, (13Z,16Z)-N,N-dimethyldocosa-13,16-dien-5-amine, (12Z,15Z)-N,N-dimethylhenicosa-12,15-dien-4-amine, (14Z,17Z)-N,N-dimethyltricosa-14,17-dien-6-amine, (15Z,18Z)-N,N-dimethyltetracosa-15,18-dien-7-amine, (18Z,21Z)-N,N-dimethylheptacosa-18,21-dien-10-amine, (15Z,18Z)-N,N-dimethyltetracosa-15,18-dien-5-amine, (14Z,17Z)-N,N-dimethyltricosa-14,17-dien-4-amine, (19Z,22Z)-N,N-dimethyloctacosa-19,22-dien-9-amine, (18Z,21Z)-N,N-dimethylheptacosa-18,21-dien-8-amine, (17Z,20Z)-N,N-dimethylhexacosa-17,20-dien-7-amine, (16Z,19Z)-N,N-dimethylpentacosa-16,19-dien-6-amine, (22Z,25Z)-N,N-dimethylhentriaconta-22,25-dien-10-amine, (21Z,24Z)-N,N-dimethyltriaconta-21,24-dien-9-amine, (18Z)-N,N-dimetylheptacos-18-en-10-amine, (17Z)-N,N-dimethylhexacos-17-en-9-amine, (19Z,22Z)-N,N-dimethyloctacosa-19,22-dien-7-amine, N,N-dimethylheptacosan-10-amine, (20Z,23Z)-N-ethyl-N-methylnonacosa-20,23-dien-10-amine, 1-[(11Z,14Z)-1-nonylicosa-11,14-dien-1-yl] pyrrolidine, (20Z)-N,N-dimethylheptacos-20-en-10-amine, (15Z)-N,N-dimethylheptacos-15-en-10-amine, (14Z)-N,N-dimethylnonacos-14-en-10-amine, (17Z)-N,N-dimethylnonacos-17-en-10-amine, (24Z)-N,N-dimethyltritriacont-24-en-10-amine, (20Z)-N,N-dimethylnonacos-20-en-10-amine, (22Z)-N,N-dimethylhentriacont-22-en-10-amine, (16Z)-N,N-dimethylpentacos-16-en-8-amine, (12Z,15Z)-N,N-dimethyl-2-nonylhenicosa-12,15-dien-1-amine, (13Z,16Z)-N,N-dimethyl-3-nonyldocosa-13,16-dien-1-amine, N,N-dimethyl-1-[(1S,2R)-2-octylcyclopropyl]heptadecan-8-amine, 1-[(1S,2R)-2-hexylcyclopropyl]-N,N-dimethylnonadecan-10-amine, N,N-dimethyl-1-[(1S ,2R)-2-octylcyclopropyl]nonadecan-10-amine, N,N-dimethyl-21-[(1S,2R)-2-octylcyclopropyl]henicosan-10-amine,N,N-dimethyl-1-[(1S,2S)-2-{[(1R,2R)-2- pentylcyclopropyl]methyl}cyclopropyl]nonadecan-10-amine,N,N-dimethyl-1-[(1S,2R)-2-octylcyclopropyl]hexadecan-8-amine, N,N-dimethyl-[(1R,2S)-2-undecylcyclopropyl]tetradecan-5-amine, N,N-dimethyl-3-{7-[(1S,2R)-2-octylcyclopropyl]heptyl} dodecan-1-amine, 1-[(1R,2S)-2-heptylcyclopropyl]-N,N- dimethyloctadecan-9-amine, 1-[(1S,2R)-2-decylcyclopropyl]-N,N-dimethylpentadecan-6-amine, N,N-dimethyl-1-[(1S,2R)-2-octylcyclopropyl]pentadecan-8-amine, R—N,N-dimethyl-1-[(9Z,12Z)-octadeca-9,12-dien-1-yloxy]-3-(octyloxy)propan-2-amine, S—N,N-dimethyl-1-[(9Z,12Z)-octadeca-9,12-dien-1-yloxy]-3-(octyloxy)propan-2-amine, 1-{2-[(9Z,12Z)-octadeca-9,12-dien-1-yloxy]-1-[(octyloxy)methyl]ethyl}pyrrolidine, (2S)-N,N-dimethyl-1-[(9Z,12Z)-octadeca-9,12-dien-1-yloxy]-3-[(5Z)-oct-5-en-1-yloxy]propan-2-amine, 1-{2-[(9Z,12Z)-octadeca-9,12-dien-1-yloxy]-1-[(octyloxy)methyl]ethyl}azetidine, (2S)-1-(hexyloxy)-N,N-dimethyl-3-[(9Z,12Z)-octadeca-9,12-dien-1-yloxy]propan-2-amine, (2S)-1-(heptyloxy)-N,N-dimethyl-3-[(9Z,12Z)-octadeca-9,12-dien-1-yloxy]propan-2-amine, N,N-dimethyl-1-(nonyloxy)-3-[(9Z,12Z)-octadeca-9,12-dien-1-yloxy]propan-2-amine, N,N-dimethyl-1-[(9Z)-octadec-9-en-1-yloxy]-3-(octyloxy)propan-2-amine; (2S)-N,N-dimethyl-1-[(6Z,9Z,12Z)-octadeca-6,9,12-trien-1-yloxy]-3-(octyloxy)propan-2-amine, (2S)-1-[(11Z,14Z)-icosa-11,14-dien-1-yloxy]-N,N-dimethyl-3-(pentyloxy)propan-2-amine, (2S)-1-(hexyloxy)-3-[(11Z,14Z)-icosa-11,14-dien-1-yloxy]-N,N-dimethylpropan-2-amine, 1-[(11Z,14Z)-icosa-11,14-dien-1-yloxy]-N,N-dimethyl-3-(octyloxy)propan-2-amine, 1-[(13Z,16Z)-docosa-13,16-dien-l-yloxy]-N,N-dimethyl-3-(octyloxy)propan-2-amine, (2S)-1-[(13Z,16Z)-docosa-13,16-dien-1-yloxy]-3-(hexyloxy)-N,N-dimethylpropan-2-amine, (2S)-1-[(13Z)-docos-13-en-1-yloxy]-3-(hexyloxy)-N,N-dimethylpropan-2-amine, 1-[(13Z)-docos-13-en-1-yloxy]-N,N-dimethyl-3-(octyloxy)propan-2-amine, 1-[(9Z)-hexadec-9-en-1-yloxy]-N,N-dimethyl-3-(octyloxy)propan-2-amine, (2R)-N,N-dimethyl-H(1-metoyloctyl)oxy]-3-[(9Z,12Z)-octadeca-9,12-dien-1-yloxy]propan-2-amine, (2R)-1-[(3,7- dimethyloctyl)oxy]-N,N-dimethyl-3-[(9Z,12Z)-octadeca-9,12-dien-1-yloxy]propan-2-amine, N,N-dimethyl-1-(octyloxy)-3-({8-[(1S,2S)-2-{[(1R,2R)-2-pentylcyclopropyl]methyl}cyclopropyl]octyl}oxy)propan-2- amine, N,N-dimethyl-1-{[8-(2-oclylcyclopropyl)octyl]oxy}-3-(octyloxy)propan-2-amine and (11E,20Z,23Z)-N,N-dimethylnonacosa-11,20,2-trien-10-amine or a pharmaceutically acceptable salt or stereoisomer thereof.

In one embodiment, a cationic lipid may be a low molecular weight cationic lipid such as those described in U.S. Publication Nos. 20130090372, 20130274504, and 20130274523.

In another embodiment, the lipid may be a cationic lipid such as, but not limited to, Formula (I) of U.S. Publication No. 20130064894. In some embodiments, the lower alkanol solution includes cationic lipids and lipid particles that provide efficient encapsulation of nucleic acids and efficient delivery of the encapsulated nucleic acid in vivo. In a non-limiting example, the cationic lipid may have Formula VIII of U.S. Publication No. 20140134260.

In another embodiment, the cationic lipid may be a trialkyl cationic lipid. Non-limiting examples of trialkyl cationic lipids and methods of making and using the trialkyl cationic lipids are described in International Patent Publication No. WO2013126803.

In one embodiment, the cationic lipid may have a positively charged hydrophilic head and a hydrophobic tail that are connected via a linker structure. As a non-limiting example, the hydrophilic head group may be primary, secondary, tertiary amines, or quaternary ammonium salts. As another non-limiting example, the lipids may have guanidino, imidazole, pyridinium, phosphorus, and arsenic groups.

In one embodiment, the cationic lipid which may be used in the formulations and delivery agents described herein may be represented by formula (I) in U.S. Publication No. 20140039032. As a non-limiting example, the cationic lipid having formula (I) in U.S. Publication No. 20140039032 may be used in a lipid nanoparticle to deliver nucleic acid molecules (e.g., polynucleotides described herein).

In one embodiment, the cationic lipid used in any of the formulations described herein may be from a class of cationic phospholipids with a highly unsaturated conjugated carontenoid fatty acid, according to the formula (I) of claim 1: Car-C(═O)-L-Cat(+) X(−), wherein Car is a carotenoid moiety, C(═O) is a carbonyl group bonded to a terminal ethenyl group of the carotenoid moiety, L is a linker bonded via an ester bond to the C(═O), linker, L optionally including a phosphate (C1-C30)alkyl ester, Cat is a cationic quaternary ammonium, and X is an anion, of International Patent Publication WO2014071072. In a non-limiting example, the formulations and delivery agents may include a liposome having this highly unsaturated conjugated carotenoid fatty acid, as described in International Patent Publication WO2014071072.

In another embodiment, the lower alkanol solution may comprise an amine cationic lipid such as those described in International Patent Publication No. WO2013059496. In one aspect, the cationic lipids may have an amino-amine or an amino-amide moiety.

In one embodiment, the cationic lipid may be synthesized by methods known in the art and/or as described in International Patent Publication Nos. WO2012040184, WO2011153120, WO2011149733, WO2011090965, WO2011043913, WO2011022460, WO2012061259, WO2012054365, WO2012044638, WO2010080724, WO201021865, WO2013086373, and WO2013086354.

In other embodiments, the lipid or lipids which may be used in the lipid nanoparticles may be, but are not limited to, 1,2-Dioleoyl-sn-glycero-3-phosphatidylcholine (DOPC), 1,2-Dioleoyl-sn-glycero-3-phosphatidylethanolamine (DOPE), cholesterol, N-[1-(2,3-Dioleyloxy)propyl]N,N,N-trimethylammonium chloride (DOTMA), 1,2-Dioleoyloxy-3-trimethylammonium-propane (DOTAP), Dioctadecylamidoglycylspermine (DOGS), N-(3-Aminopropyl)-N,N-dimethyl-2,3-bis(dodecyloxy)-1-propanaminium bromide (GAP-DLRIE), cetyltrimethylammonium bromide (CTAB), 6-lauroxyhexyl ornithinate (LHON), 1-(2,3-Dioleoloxypropyl)2,4,6-trimethylpyridinium (2Oc), 2,3-Dioleyloxy-N-[2(sperminecarboxamido)-ehtyl]-N,N-dimethyl-1- propanaminium trifluoroacetate (DOSPA), 1,2-Dioleyl-3-trimethylammonium-propane (DOPA), N-(2-Hydroxyethyl)-N,N-dimethyl-2,3-bis(tetradecyloxy)-1-propanaminium bromide (MDRIE), Dimyristooxypropyl dimethyl hydroxyethyl ammonium bromide (DMRI), 3β-[N-(N′,N′-Dimethylaminoethane)-carbamoyl]cholesterol (DC-Chol), Bis-guanidium-tren-cholesterol (BGTC), 1,3-Dioleoxy-2-(6-carboxy-spermyl)-propylamide (DOSPER), Dimethyloctadecylammonium bromide (DDAB), Dioctadecylamidoglicylspermidin (DSL), rac-[(2,3-Dioctadecyloxypropyl)(2-hydroxyethyl)]-dimethylammonium chloride (CLIP-1), rac-[2(2,3-Dihexadecyloxypropyl-oxymethyloxy)ehtyl]trimethylammonium chloride (CLIP-6), Ethyldimyrisotylphosphatidylcholine (EDMPC), 1,2-Distearyloxy-N,N-dimethyl-3-aminopropane (DSDMA), 1,2-Dimyristoyl-trimethylammoniumpropane (DMTAP), O,O′-Dimyristyl-N-lysyl asparate (DMKE), 1,2-Distearoyl-sn-glycero-3-ethylphosphocholine (DSEPC), N-Palmitoyl-D-erythro-spingosyl carbamoyl-spermine (CCS), N-t-Butyl-No-tetradecyl-3-tetradecylaminopropionamidine (diC14-amidine), Octadecenolyoxy[ethyl-2-heptadecenyl-3 hydroxyethyl] imidazolinium chloride (DOTIM), N1-Cholesteryloxycarbonyl-3,7-diazanonane-1,9-diamine (CDAN) and 2-(3-[Bis-(3-amino-propyl)-amino]propylamino)-N- ditetradecylcarbamoylme-ethyl-acetamide (RPR2091290).

In one embodiment, the lipids may be a cleavable lipid. As a non-limiting example, the cleavable lipid and/or pharmaceutical compositions comprising cleavable lipids may be those described in International Publication No. WO2012170889. As another non-limiting example, the cleavable lipid may be HGT4001, HGT4002, HGT4003, HGT4004 and/or HGT4005 as described in International Patent Publication No. WO2012170889.

In some embodiments, polyethylene glycol (PEG) is included as part of the lipid components in the lower alkanol solution. The ratio of PEG in the lipid nanoparticle (LNP) formulations may be increased or decreased and/or the carbon chain length of the PEG lipid may be modified from C14 to C18 to alter the pharmacokinetics and/or biodistribution of the lipid nanoparticle formulations. As a non-limiting example, lipid nanoparticle formulations may contain from about 0.5% to about 3.0%, from about 1.0% to about 3.5%, from about 1.5% to about 4.0%, from about 2.0% to about 4.5%, from about 2.5% to about 5.0%, or from about 3.0% to about 6.0% of the lipid molar ratio of PEG-c-DOMG, as compared to the lipid, DSPC and cholesterol. In another embodiment the PEG-c-DOMG may be replaced with a PEG lipid such as, but not limited to, PEG-DSG (1,2-Distearoyl-sn-glycerol, methoxypolyethylene glycol), PEG-DMG (1,2-Dimyristoyl-sn-glycerol) and/or PEG-DPG (1,2-Dipalmitoyl-sn-glycerol, methoxypolyethylene glycol).

In one embodiment, the lipid nanoparticle includes a lipid formulation including 45-65 mol % of a lipid (e.g., either cationic lipid or an ionizable lipid), 5 mol % to about 10 mol %, of a non-cationic lipid of overall neutral charge, 25-40 mol % of a sterol, and 0.5-5 mol % of a PEG or PEG-modified lipid. Non-limiting examples of nucleic acid particles are disclosed in U.S. Publication No 20140121263.

In some embodiments, lower alkanol solution includes the lipid KL52 (an amino-lipid disclosed in U.S. Publication No. 20120295832). Activity and/or safety (as measured by examining one or more of ALT/AST, white blood cell count and cytokine induction) of lipid nanoparticle administration may be improved by incorporation of such lipids. Lipid nanoparticles including KL52 may be administered intravenously and/or in one or more doses. In some embodiments, administration of lipid nanoparticles including KL52 results in equal or improved mRNA and/or protein expression as compared to lipid nanoparticles including MC3.

In some embodiments, the lower alkanol solution may include linear amino-lipids as described in U.S. Pat. No. 8,691,750.

In some embodiments, the lower alkanol solution includes heptatriaconta-6,9,28,31-tetraen-19-yl-4-(dimethylamino)butanoate (DLin-MC3-DMA), phosphatidylcholine (1,2-distearoyl-sn-glycero-3-phosphocholine (DSPC), cholesterol, and polyethylene glycol-dimyristolglycerol (PEG-DMG). The ratio of DLin-MC3-DMA:DSPC:Cholesterol:PEG-DMG can be about 50:10:38.5:1.5.

Solvents that can be used as part of the lower alkanol solution include any alcohol with six or fewer carbon atoms, e.g., methanol, ethanol, propanol, pentanol, and isomers and mixtures thereof.

### Aqueous Solution

An aqueous solution of the present invention serves as a dispersant for the lipid nanoparticles and, additionally, provides hydrophilic encapsulants (e.g., nucleic acid). The aqueous solution can be nucleic acid suspended in water, or the aqueous solution can be a buffered solution, such as a citrate buffer (e.g., 100 mM citrate buffer), a phosphate buffer solution (e.g., phosphate buffered saline (PBS)), or a TRIS buffer (e.g., TRIS/sucrose).

In some embodiments, the aqueous solution has a pH from about 3.0 to about 8.0 (e.g., about 3.0, about 3.1, about 3.2, about 3.3, about 3.4, about 3.5, about 3.6, about 3.7, about 3.8, about 3.9, about 4.0, about 4.1, about 4.2, about 4.3, about 4.4, about 4.5, about 4.6, about 4.7, about 4.8, about 4.9, about 5.0, about 5.1, about 5.2, about 5.3, about 5.4, about 5.5, about 5.6, about 5.7, about 5.8, about 5.9, about 6.0, about 6.1, about 6.2, about 6.3, about 6.4, about 6.5, about 6.6, about 6.7, about 6.8, about 6.9, about 7.0, about 7.1, about 7.2, about 7.3, about 7.4, about 7.5, about 7.6, about 7.7, about 7.9, or about 8.0).

The aqueous solution can have an osmolality from about 200 mOsm to about 400 mOsm (e.g., from about 250 mOsm to about 350 mOsm, or about 260 mOsm, 270 mOsm, 280 mOsm, 290 mOsm, 300 mOsm, 310 mOsm, 320 mOsm, 330 mOsm, 340 mOsm, or 350 mOsm).

Hydrophilic encapsulants that can be dissolved or dispersed in the aqueous solution of the invention include nucleic acid (e.g., DNA, RNA, e.g., mRNA).

The concentration of encapsulants in the aqueous solution (e.g., dry mass of encapsulant per unit volume of liquid, e.g., aqueous liquid, e.g., buffer) will depend on various parameters and may be between 1 μg/ml and 100,000 μg/ml (e.g., between 1 μg/ml and 50,000 μg/ml, between 2 μg/ml and 10,000 μg/ml, between 5 μg/ml and 2,000 μg/ml, between 10 μg/ml and 1,500 μg/ml, between 25 μg/ml and 1,000 μg/ml, between 50 μg/ml and 200 μg/ml, between 50 μg/ml and 500 μg/ml, or between 100 μg/ml and 200 μg/ml, e.g., about 1 μg/ml, about 5 μg/ml, about 10 μg/ml, about 20 μg/ml, about 30 μg/ml, about 40 μg/ml, about 50 μg/ml, about 60 μg/ml, about 70 μg/ml, about 80 μg/ml, about 90 μg/ml, about 100 μg/ml, about 110 μg/ml, about 111 μg/ml, about 111.11 μg/ml, about 120 μg/ml, about 130 μg/ml, about 140 μg/ml, about 150 μg/ml, about 175 μg/ml, about 200 μg/ml, about 300 μg/ml, about 400 μg/ml, or about 500 μg/ml). In some embodiments, 450 μl of aqueous solution contains 50 μg encapsulant (e.g., mRNA). Additionally or alternatively, 900 μl of aqueous solution contains 100 μg encapsulant (e.g., mRNA). Additionally or alternatively, 1,800 μl of aqueous solution contains 200 μg encapsulant (e.g., mRNA).

Nucleotides, nucleosides, or polynucleotides (e.g., mRNA) of the invention may include alterations (e.g., various distinct alterations). In some embodiments, where the polynucleotide is an mRNA, the coding region, the flanking regions and/or the terminal regions (e.g., a 3′-stabilizing region) may contain one, two, or more (optionally different) nucleoside or nucleotide alterations. In some embodiments, an alternative polynucleotide introduced to a cell may exhibit reduced degradation in the cell, as compared to an unaltered polynucleotide.

The polynucleotides of the invention can include any useful alteration, such as to the nucleobase, the sugar, or the internucleoside linkage (e.g., to a linking phosphate/to a phosphodiester linkage/to the phosphodiester backbone). In certain embodiments, alterations (e.g., one or more alterations) are present in each of the nucleobase, the sugar, and the internucleoside linkage. Alterations according to the present invention may be alterations of ribonucleic acids (RNAs) to deoxyribonucleic acids (DNAs), e.g., the substitution of the 2′-OH of the ribofuranosyl ring to 2′-H, threose nucleic acids (TNAs), glycol nucleic acids (GNAs), peptide nucleic acids (PNAs), locked nucleic acids (LNAs), or hybrids thereof. Additional alterations are described herein.

As described herein, in some embodiments, the polynucleotides of the invention do not substantially induce an innate immune response of a cell into which the polynucleotide (e.g., mRNA) is introduced. Features of an induced innate immune response include 1) increased expression of pro-inflammatory cytokines, 2) activation of intracellular PRRs (RIG-I, MDA5, etc, and/or 3) termination or reduction in protein translation.

The polynucleotides can optionally include other agents (e.g., RNAi-inducing agents, RNAi agents, siRNAs, shRNAs, miRNAs, antisense RNAs, ribozymes, catalytic DNA, tRNA, RNAs that induce triple helix formation, aptamers, vectors). In some embodiments, the polynucleotides may include one or more messenger RNAs (mRNAs) having one or more alternative nucleoside or nucleotides (i.e., alternative mRNA molecules). Details for these polynucleotides follow.

### Nucleobase Alternatives

The alternative nucleosides and nucleotides can include an alternative nucleobase. Examples of nucleobases found in RNA include, but are not limited to, adenine, guanine, cytosine, and uracil. Examples of nucleobases found in DNA include, but are not limited to, adenine, guanine, cytosine, and thymine. These nucleobases can be altered or wholly replaced to provide polynucleotide molecules having enhanced properties, e.g., increased stability such as resistance to nucleases.

Alternative nucleotide base pairing encompasses not only the standard adenine-thymine, adenine-uracil, or guanine-cytosine base pairs, but also base pairs formed between nucleotides and/or alternative nucleotides including non-standard or alternative bases, wherein the arrangement of hydrogen bond donors and hydrogen bond acceptors permits hydrogen bonding between a non-standard base and a standard base or between two complementary non-standard base structures. One example of such non-standard base pairing is the base pairing between the alternative nucleotide inosine and adenine, cytosine, or uracil.

In some embodiments, the nucleobase is an alternative uracil. Exemplary nucleobases and nucleosides having an alternative uracil include pseudouridine (ψ), pyridin-4-one ribonucleoside, 5-aza-uracil, 6-aza-uracil, 2-thio-5-aza-uracil, 2-thio-uracil (s²U), 4-thio-uracil (s⁴U), 4-thio-pseudouridine, 2-thio-pseudouridine, 5-hydroxy-uracil (ho⁵U), 5-aminoallyl-uracil, 5-halo-uracil (e.g., 5-iodo-uracil or 5-bromo-uracil), 3-methyl-uracil (m³U), 5-methoxy-uracil (mo⁵U), uracil 5-oxyacetic acid (cmo⁵U), uracil 5-oxyacetic acid methyl ester (mcmo⁵U), 5-carboxymethyl-uracil (cm⁵U), 1-carboxymethyl-pseudouridine, 5-carboxyhydroxymethyl-uracil (chm⁵U), 5-carboxyhydroxymethyl-uracil methyl ester (mchm⁵U), 5-methoxycarbonylmethyl-uracil (mcm⁵U), 5-methoxycarbonylmethyl-2-thio-uracil (mcm⁵s²U), 5-aminomethyl-2-thio-uracil (nm⁵s²U), 5-methylaminomethyl-uracil (mnm⁵U), 5-methylaminomethyl-2-thio-uracil (mnm⁵s²U), 5-methylaminomethyl-2-seleno-uracil (mnm⁵se²U), 5-carbamoylmethyl-uracil (ncm⁵U), 5-carboxymethylaminomethyl-uracil (cmnm⁵U), 5-carboxymethylaminomethyl-2-thio-uracil (cmnm⁵s²U), 5-propynyl-uracil, 1-propynyl-pseudouracil, 5-taurinomethyl-uracil (τm⁵U), 1-taurinomethyl-pseudouridine, 5-taurinomethyl-2-thio-uracil(τm⁵s²U), 1-taurinomethyl-4-thio-pseudouridine, 5-methyl-uracil (m⁵U, i.e., having the nucleobase deoxythymine), 1-methyl-pseudouridine (m¹ψ), 5-methyl-2-thio-uracil (m⁵s²U), 1-methyl-4-thio-pseudouridine (m¹s⁴ψ) 4-thio-1-methyl-pseudouridine, 3-methyl-pseudouridine (m³ψ), 2-thio-1-methyl-pseudouridine, 1-methyl-1-deaza-pseudouridine, 2-thio-1-methyl-1-deaza-pseudouridine, dihydrouracil (D), dihydropseudouridine, 5,6-dihydrouracil, 5-methyl-dihydrouracil (m⁵D), 2-thio-dihydrouracil, 2-thio-dihydropseudouridine, 2-methoxy-uracil, 2-methoxy-4-thio-uracil, 4-methoxy-pseudouridine, 4-methoxy-2-thio-pseudouridine, N1-methyl-pseudouridine, 3-(3-amino-3-carboxypropyl)uracil (acp³U), 1-methyl-3-(3-amino-3-carboxypropyl)pseudouridine (acp³ψ), 5-(isopentenylaminomethyl)uracil (inm⁵U), 5-(isopentenylaminomethyl)-2-thio-uracil (inm⁵s²U), 5,2′-O-dimethyl-uridine (m⁵Um), 2-thio-2′-O-methyl-uridine (s²Um), 5-methoxycarbonylmethyl-2′-O-methyl-uridine (mcm⁵Um), 5-carbamoylmethyl-2′-O-methyl-uridine (ncm⁵Um), 5-carboxymethylaminomethyl-2′-O-methyl-uridine (cmnm⁵Um), 3,2′-O-dimethyl-uridine (m³Um), and 5-(isopentenylaminomethyl)-2′-O-methyl-uridine (inm⁵Um), 1-thio-uracil, deoxythymidine, 5-(2-carbomethoxyvinyl)-uracil, 5-(carbamoylhydroxymethyl)-uracil, 5-carbamoylmethyl-2-thio-uracil, 5-carboxymethyl-2-thio-uracil, 5-cyanomethyl-uracil, 5-methoxy-2-thio-uracil, and 5-[3-(1-E-propenylamino)]uracil.

In some embodiments, the nucleobase is an alternative cytosine. Exemplary nucleobases and nucleosides having an alternative cytosine include 5-aza-cytosine, 6-aza-cytosine, pseudoisocytidine, 3-methyl-cytosine (m3C), N4-acetyl-cytosine (ac4C), 5-formyl-cytosine (f5C), N4-methyl-cytosine (m4C), 5-methyl-cytosine (m5C), 5-halo-cytosine (e.g., 5-iodo-cytosine), 5-hydroxymethyl-cytosine (hm5C), 1-methyl-pseudoisocytidine, pyrrolo-cytosine, pyrrolo-pseudoisocytidine, 2-thio-cytosine (s2C), 2-thio-5-methyl-cytosine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytosine, 2-methoxy-5-methyl-cytosine, 4-methoxy-pseudoisocytidine, 4-methoxy-1-methyl-pseudoisocytidine, lysidine (k2C), 5,2′-O-dimethyl-cytidine (m5Cm), N4-acetyl-2′-O-methyl-cytidine (ac4Cm), N4,2′-O-dimethyl-cytidine (m4Cm), 5-formyl-2′-O-methyl-cytidine (f5Cm), N4,N4,2′-O-trimethyl-cytidine (m42Cm), 1-thio-cytosine, 5-hydroxy-cytosine, 5-(3-azidopropyl)-cytosine, and 5-(2-azidoethyl)-cytosine.

In some embodiments, the nucleobase is an alternative adenine. Exemplary nucleobases and nucleosides having an alternative adenine include 2-amino-purine, 2,6-diaminopurine, 2-amino-6-halo-purine (e.g., 2-amino-6-chloro-purine), 6-halo-purine (e.g., 6-chloro-purine), 2-amino-6-methyl-purine, 8-azido-adenine, 7-deaza-adenine, 7-deaza-8-aza-adenine, 7-deaza-2-amino-purine, 7-deaza-8-aza-2-amino-purine, 7-deaza-2,6-diaminopurine, 7-deaza-8-aza-2,6-diaminopurine, 1-methyl-adenine (m1A), 2-methyl-adenine (m2A), N6-methyl-adenine (m6A), 2-methylthio-N6-methyl-adenine (ms2m6A), N6-isopentenyl-adenine (i6A), 2-methylthio-N6-isopentenyl-adenine (ms2i6A), N6-(cis-hydroxyisopentenyl)adenine (io6A), 2-methylthio-N6-(cis-hydroxyisopentenyl)adenine (ms2io6A), N6-glycinylcarbamoyl-adenine (g6A), N6-threonylcarbamoyl-adenine (t6A), N6-methyl-N6-threonylcarbamoyl-adenine (m6t6A), 2-methylthio-N6-threonylcarbamoyl-adenine (ms2g6A), N6,N6-dimethyl-adenine (m62A), N6-hydroxynorvalylcarbamoyl-adenine (hn6A), 2-methylthio-N6-hydroxynorvalylcarbamoyl-adenine (ms2hn6A), N6-acetyl-adenine (ac6A), 7-methyl-adenine, 2-methylthio-adenine, 2-methoxy-adenine, N6,2′-O-dimethyl-adenosine (m6Am), N6,N6,2′-O-trimethyl-adenosine (m62Am), 1,2′-O-dimethyl-adenosine (m1Am), 2-amino-N6-methyl-purine, 1-thio-adenine, 8-azido-adenine, N6-(19-amino-pentaoxanonadecyl)-adenine, 2,8-dimethyl-adenine, N6-formyl-adenine, and N6-hydroxymethyl-adenine.

In some embodiments, the nucleobase is an alternative guanine. Exemplary nucleobases and nucleosides having an alternative guanine include inosine (I), 1-methyl-inosine (m1I), wyosine (imG), methylwyosine (mimG), 4-demethyl-wyosine (imG-14), isowyosine (imG2), wybutosine (yW), peroxywybutosine (o2yW), hydroxywybutosine (OHyW), undermodified hydroxywybutosine (OHyW*), 7-deaza-guanine, queuosine (Q), epoxyqueuosine (oQ), galactosyl-queuosine (galQ), mannosyl-queuosine (manQ), 7-cyano-7-deaza-guanine (preQ0), 7-aminomethyl-7-deaza-guanine (preQ1), archaeosine (G+), 7-deaza-8-aza-guanine, 6-thio-guanine, 6-thio-7-deaza-guanine, 6-thio-7-deaza-8-aza-guanine, 7-methyl-guanine (m7G), 6-thio-7-methyl-guanine, 7-methyl-inosine, 6-methoxy-guanine, 1-methyl-guanine (m1G), N2-methyl-guanine (m2G), N2,N2-dimethyl-guanine (m22G), N2,7-dimethyl-guanine (m2,7G), N2, N2,7-dimethyl-guanine (m2,2,7G), 8-oxo-guanine, 7-methyl-8-oxo-guanine, 1-methyl-6-thio-guanine, N2-methyl-6-thio-guanine, N2,N2-dimethyl-6-thio-guanine, N2-methyl-2′-O-methyl-guanosine (m2Gm), N2,N2-dimethyl-2′-O-methyl-guanosine (m22Gm), 1-methyl-2′-O-methyl-guanosine (m1Gm), N2,7-dimethyl-2′-O-methyl-guanosine (m2,7Gm), 2′-O-methyl-inosine (Im), 1,2′-O-dimethyl-inosine (m1Im), 1-thio-guanine, and O-6-methyl-guanine.

The alternative nucleobase of a nucleotide can be independently a purine, a pyrimidine, a purine or pyrimidine analog. For example, the nucleobase can be an alternative to adenine, cytosine, guanine, uracil, or hypoxanthine. In another embodiment, the nucleobase can also include, for example, naturally-occurring and synthetic derivatives of a base, including pyrazolo[3,4-d]pyrimidines, 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-propynyl uracil and cytosine, 6-azo uracil, cytosine and thymine, 5-uracil (pseudouracil), 4-thiouracil, 8-halo (e.g., 8-bromo), 8-amino, 8-thiol, 8-thioalkyl, 8-hydroxy and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, deazaguanine, 7-deazaguanine, 3-deazaguanine, deazaadenine, 7-deazaadenine, 3-deazaadenine, pyrazolo[3,4-d]pyrimidine, imidazo[1,5-a]1,3,5 triazinones, 9-deazapurines, imidazo[4,5-d]pyrazines, thiazolo[4,5-d]pyrimidines, pyrazin-2-ones, 1,2,4-triazine, pyridazine; or 1,3,5 triazine. When the nucleotides are depicted using the shorthand A, G, C, T or U, each letter refers to the representative base and/or derivatives thereof, e.g., A includes adenine or adenine analogs, e.g., 7-deaza adenine).

### Alterations on the Sugar

The alternative nucleosides and nucleotides, which may be incorporated into a polynucleotide of the invention (e.g., RNA or mRNA, as described herein), can be altered on the sugar of the nucleoside or nucleotide. In some embodiments, the alternative nucleosides or nucleotides include the structure:

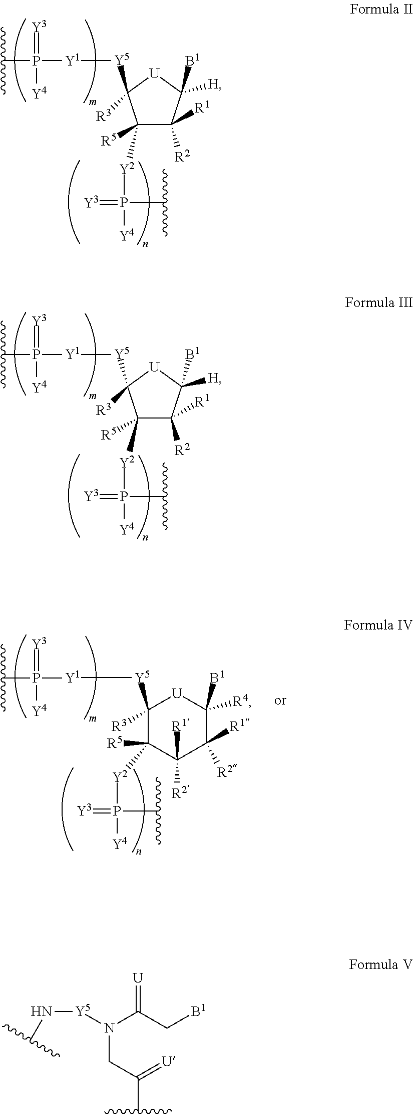

wherein B1 is a nucleobase;

each U and U′ is, independently, O, S, N(RU)nu, or C(RU)nu, wherein nu is 1 or 2 (e.g., 1 for N(RU)nu and 2 for C(RU)nu) and each RU is, independently, H, halo, or optionally substituted C1-C6 alkyl;

each of R1, R1′, R1″, R2, R2′, R2″, R3, R4, and R5 is, independently, H, halo, hydroxy, thiol, optionally substituted C1-C6 alkyl, optionally substituted C2-C6 alkynyl, optionally substituted C1-C6 heteroalkyl, optionally substituted C2-C6 heteroalkenyl, optionally substituted C2-C6 heteroalkynyl, optionally substituted amino, azido, optionally substituted C6-C10 aryl; or R3 and/or R5 can join together with one of R1, R1′, R1″, R2, R2′, or R2″ to form together with the carbons to which they are attached an optionally substituted C3-C10 carbocycle or an optionally substituted C3-C9 heterocyclyl;

each of m and n is independently, 0, 1, 2, 3, 4, or 5;

each of Y1, Y2, and Y3, is, independently, O, S, Se, —NRN1-, optionally substituted C1-C6 alkylene, or optionally substituted C1-C6 heteroalkylene, wherein RN1 is H, optionally substituted C1-C6 alkyl, optionally substituted C2-C6 alkenyl, optionally substituted C2-C6 alkynyl, or optionally substituted C6-C10 aryl; and

each Y4 is, independently, H, hydroxy, protected hydroxy, halo, thiol, boranyl, optionally substituted C1-C6 alkyl, optionally substituted C2-C6 alkenyl, optionally substituted C2-C6 alkynyl, optionally substituted C1-C6 heteroalkyl, optionally substituted C2-C6 heteroalkenyl, optionally substituted C2-C6 heteroalkynyl, or optionally substituted amino; and

Y5 is O, S, Se, optionally substituted C1-C6 alkylene, or optionally substituted C1-C6 heteroalkylene;

or a salt thereof.

In some embodiments, the 2′-hydroxy group (OH) can be modified or replaced with a number of different substituents. Exemplary substitutions at the 2′-position include, but are not limited to, H, azido, halo (e.g., fluoro), optionally substituted C₁₋₆ alkyl (e.g., methyl); optionally substituted C₁₋₆ alkoxy (e.g., methoxy or ethoxy); optionally substituted C₆₋₁₀ aryloxy; optionally substituted C₃₋₈ cycloalkyl; optionally substituted C₆₋₁₀ aryl-C₁₋₆ alkoxy, optionally substituted C₁₋₁₂ (heterocyclyl)oxy; a sugar (e.g., ribose, pentose, or any described herein); a polyethyleneglycol (PEG), —O(CH₂CH₂O)ₙCH₂CH₂OR, where R is H or optionally substituted alkyl, and n is an integer from 0 to 20 (e.g., from 0 to 4, from 0 to 8, from 0 to 10, from 0 to 16, from 1 to 4, from 1 to 8, from 1 to 10, from 1 to 16, from 1 to 20, from 2 to 4, from 2 to 8, from 2 to 10, from 2 to 16, from 2 to 20, from 4 to 8, from 4 to 10, from 4 to 16, and from 4 to 20); “locked” nucleic acids (LNA) in which the 2′-hydroxy is connected by a C₁₋₆ alkylene or C₁₋₆ heteroalkylene bridge to the 4′-carbon of the same ribose sugar, where exemplary bridges included methylene, propylene, ether, or amino bridges; aminoalkyl, as defined herein; aminoalkoxy, as defined herein; amino as defined herein; and amino acid, as defined herein.

Generally, RNA includes the sugar group ribose, which is a 5-membered ring having an oxygen. Exemplary, non-limiting alternative nucleotides include replacement of the oxygen in ribose (e.g., with S, Se, or alkylene, such as methylene or ethylene); addition of a double bond (e.g., to replace ribose with cyclopentenyl or cyclohexenyl); ring contraction of ribose (e.g., to form a 4-membered ring of cyclobutane or oxetane); ring expansion of ribose (e.g., to form a 6- or 7-membered ring having an additional carbon or heteroatom, such as for anhydrohexitol, altritol, mannitol, cyclohexanyl, cyclohexenyl, and morpholino (that also has a phosphoramidate backbone)); multicyclic forms (e.g., tricyclo and “unlocked” forms, such as glycol nucleic acid (GNA) (e.g., R-GNA or S-GNA, where ribose is replaced by glycol units attached to phosphodiester bonds), threose nucleic acid (TNA, where ribose is replace with α-L-threofuranosyl-(3′→2′)), and peptide nucleic acid (PNA, where 2-amino-ethyl-glycine linkages replace the ribose and phosphodiester backbone).

In some embodiments, the sugar group contains one or more carbons that possess the opposite stereochemical configuration of the corresponding carbon in ribose. Thus, a polynucleotide molecule can include nucleotides containing, e.g., arabinose or L-ribose, as the sugar.

In some embodiments, the polynucleotide of the invention includes at least one nucleoside wherein the sugar is L-ribose, 2′-O-methyl-ribose, 2′-fluoro-ribose, arabinose, hexitol, an LNA, or a PNA.

### Alterations on the Internucleoside Linkage

The alternative nucleotides, which may be incorporated into a polynucleotide of the invention, can be altered on the internucleoside linkage (e.g., phosphate backbone). Herein, in the context of the polynucleotide backbone, the phrases “phosphate” and “phosphodiester” are used interchangeably. Backbone phosphate groups can be altered by replacing one or more of the oxygen atoms with a different substituent.

The alternative nucleotides can include the wholesale replacement of an unaltered phosphate moiety with another internucleoside linkage as described herein. Examples of alternative phosphate groups include, but are not limited to, phosphorothioate, phosphoroselenates, boranophosphates, boranophosphate esters, hydrogen phosphonates, phosphoramidates, phosphorodiamidates, alkyl or aryl phosphonates, and phosphotriesters. Phosphorodithioates have both non-linking oxygens replaced by sulfur. The phosphate linker can also be altered by the replacement of a linking oxygen with nitrogen (bridged phosphoramidates), sulfur (bridged phosphorothioates), and carbon (bridged methylene-phosphonates).

The alternative nucleosides and nucleotides can include the replacement of one or more of the non-bridging oxygens with a borane moiety (BH₃), sulfur (thio), methyl, ethyl, and/or methoxy. As a non-limiting example, two non-bridging oxygens at the same position (e.g., the alpha (α), beta (β) or gamma (γ) position) can be replaced with a sulfur (thio) and a methoxy.

The replacement of one or more of the oxygen atoms at the a position of the phosphate moiety (e.g., α-thio phosphate) is provided to confer stability (such as against exonucleases and endonucleases) to RNA and DNA through the unnatural phosphorothioate backbone linkages. Phosphorothioate DNA and RNA have increased nuclease resistance and subsequently a longer half-life in a cellular environment.

Other internucleoside linkages that may be employed according to the present invention, including internucleoside linkages which do not contain a phosphorous atom, are described herein.

### Lipid Nanoparticle Compositions

The present invention provides lipid nanoparticles produced by injecting a lower alkanol solution into an aqueous solution having a lipid, for example, by injecting at an automated rate. For example, lipid nanoparticles of the present invention can be produced by any of the methods and/or apparatus of the invention.

In some embodiments, the injection of a lower alkanol solution into an aqueous solution is about 1,000 to about 5,000 microliters per second (μl/s; e.g., from about 1,000 to about 5,000 μl/s, from about 1,500 to about 4,500 μl/s, from about 2,000 to about 4,000 μl/s, or from about 2,500 to about 3,000 μl/s). In some embodiments, the injecting is at a rate of 2,600 μl/s.

In some embodiments, the ratio of lipid to nucleic acid, e.g., mRNA or alternative mRNA, in the lipid nanoparticle may be from about 5:1 to about 10:1, from about 5:1 to about 15:1, from about 5:1 to about 20:1, from about 5:1 to about 25:1, from about 5:1 to about 30:1, from about 5:1 to about 35:1, from about 5:1 to about 40:1, from about 5:1 to about 45:1, from about 5:1 to about 50:1, from about 5:1 to about 55:1, from about 5:1 to about 60:1, from about 5:1 to about 70:1, from about 10:1 to about 15:1, from about 10:1 to about 20:1, from about 10:1 to about 25:1, from about 10:1 to about 30:1, from about 10:1 to about 35:1, from about 10:1 to about 40:1, from about 10:1 to about 45:1, from about 10:1 to about 50:1, from about 10:1 to about 55:1, from about 10:1 to about 60:1, from about 10:1 to about 70:1, from about 15:1 to about 20:1, from about 15:1 to about 25:1,from about 15:1 to about 30:1, from about 15:1 to about 35:1, from about 15:1 to about 40:1, from about 15:1 to about 45:1, from about 15:1 to about 50:1, from about 15:1 to about 55:1, from about 15:1 to about 60:1 or from about 15:1 to about 70:1.

In one embodiment, the zeta potential of the lipid nanoparticles is from about −100 mV to about +100 mV (e.g., from −80 mV to +80 mV, from −50 mV to +50 mV, from −40 mV to +40 mV, from −30 mV to +30 mV, from −20 mV to +20 mV, from −10 mV to +10 mV, e.g., about −100 mV, about −90 mV, about −80 mV, about −70 mV, about −60 mV, about −50 mV, about −40 mV, about −30 mV, about −20 mV, about −10 mV, about 0 mV, about +10 mV, about +20 mV, about +30 mV, about +40 mV, about +50 mV, about +60 mV, about +70 mV, about +80 mV, about +90 mV, or about +100 mV) at a pH in the range of 6-8.

Lipid nanoparticle formulations can include biodegradable cationic lipids to produce nanoparticles known as a rapidly eliminated lipid nanoparticle (reLNP). Ionizable cationic lipids, such as, but not limited to, DLinDMA, DLin-KC2-DMA, and DLin-MC3-DMA, have been shown to accumulate in plasma and tissues over time and may be a potential source of toxicity. The rapid metabolism of the rapidly eliminated lipids can improve the tolerability and therapeutic index of the lipid nanoparticles by an order of magnitude from a 1 mg/kg dose to a 10 mg/kg dose in rat. Inclusion of an enzymatically degraded ester linkage can improve the degradation and metabolism profile of the cationic component, while still maintaining the activity of the reLNP formulation. The ester linkage can be internally located within the lipid chain or it may be terminally located at the terminal end of the lipid chain. The internal ester linkage may replace any carbon in the lipid chain.

In one embodiment, the lipid nanoparticles described herein may include 50% DLin-KC2-DMA, 10% DSPC, 39.5% cholesterol and 0.5% PEG-DSG. In one embodiment, the lipid nanoparticles described herein may include 50% DLin-KC2-DMA, 10% DSPC, 39.5% cholesterol and 0.5% PEG-DSPE.

In one embodiment, the lipid nanoparticles described herein may include 50% DLin-KC2-DMA, 10% DSPC, 38.5% cholesterol and 1.5% PEG-DSG. In one embodiment, the lipid nanoparticles described herein may include 50% DLin-KC2-DMA, 10% DSPC, 38.5% cholesterol and 1.5% PEG-DSPE.

In one embodiment, the lipid nanoparticles described herein may include 50% DLin-KC2-DMA, 10% DSPC, 35% cholesterol and 5% PEG-DSG. In one embodiment, the lipid nanoparticles described herein may include 50% DLin-KC2-DMA, 10% DSPC, 35% cholesterol and 5% PEG-DSPE.

In one embodiment, the lipid nanoparticles described herein may include 50% DLin-KC2-DMA, 10% DSPC, 39.5% cholesterol and 0.5% PEG-DSG. In one embodiment, the lipid nanoparticles described herein may include 50% DLin-KC2-DMA, 10% DSPC, 30% cholesterol and 10% PEG-DSPE.

Lipid nanoparticles may also include lipidoids. The synthesis of lipidoids has been extensively described and formulations containing these compounds are particularly suited for delivery of polynucleotides (see Mahon et al., Bioconjug Chem. 2010 21:1448-1454; Schroeder et al., J Intern Med. 2010 267:9-21; Akinc et al., Nat Biotechnol. 2008 26:561-569; Love et al., Proc Natl Acad Sci USA. 2010 107:1864-1869; Siegwart et al., Proc Natl Acad Sci USA. 2011 108:12996-3001).

The lipidoid referred to herein as “98N12-5” is disclosed by Akinc et al., Mol Ther 2009 17:872-879. The lipidoid referred to herein as “C12-200” is disclosed by Love et al., Proc Natl Acad Sci USA. 2010 107:1864-1869 and Liu and Huang, Mol Ther 2010 669-670. The lipidoid formulations can include particles including either 3 or 4 or more components in addition to polynucleotides. As an example, formulations with certain lipidoids, include, but are not limited to, 98N12-5 and may contain 42% lipidoid, 48% cholesterol and 10% PEG (C14 alkyl chain length). As another example, formulations with certain lipidoids, include, but are not limited to, C12-200 and may contain 50% lipidoid, 10% disteroylphosphatidyl choline, 38.5% cholesterol, and 1.5% PEG-DMG.

In one embodiment, a polynucleotide formulated with a lipidoid for systemic intravenous administration can target the liver. For example, a final optimized intravenous formulation using polynucleotides, and including a lipid molar composition of 42% 98N12-5, 48% cholesterol, and 10% PEG-lipid with a final weight ratio of about 7.5 to 1 total lipid to polynucleotides, and a C14 alkyl chain length on the PEG lipid, with a mean or average particle size of roughly 50-60 nm, can result in the distribution of the formulation to be greater than 90% to the liver (see, Akinc et al, Mol Ther 2009 17:872-879). In another example, an intravenous formulation using a C12-200 (see U.S. Pat. No. 8,883,202 and International Patent Publication WO2010129709) lipidoid may have a molar ratio of 50/10/38.5/1.5 of C12-200/disteroylphosphatidyl choline/cholesterol/PEG-DMG, with a weight ratio of 7 to 1 total lipid to polynucleotides, and a mean or average particle size of 80 nm may be effective to deliver polynucleotides to hepatocytes (see, Love et al., Proc Natl Aced Sci USA. 2010 107:1864-1869). In another embodiment, an MD1 lipidoid-containing formulation may be used to effectively deliver polynucleotides to hepatocytes in vivo.

The characteristics of optimized lipidoid formulations for intramuscular or subcutaneous routes may vary significantly depending on the target cell type and the ability of formulations to diffuse through the extracellular matrix into the blood stream. While an average particle size of less than 150 nm may be desired for effective hepatocyte delivery due to the size of the endothelial fenestrae (see, Akinc et al., Mol Ther. 2009 17:872-879), use of a lipidoid-formulated polynucleotides to deliver the formulation to other cells types including, but not limited to, endothelial cells, myeloid cells, and muscle cells may not be similarly size-limited.

Use of lipidoid formulations to deliver siRNA in vivo to other non-hepatocyte cells such as myeloid cells and endothelium has been reported (see Akinc et al., Nat Biotechnol. 2008 26:561-569; Leuschner et al., Nat Biotechnol. 2011 29:1005-1010; Cho et al. Adv. Funct. Mater. 2009 19:3112-3118; 8th International Judah Folkman Conference, Cambridge, Mass. Oct. 8-9, 2010). Effective delivery to myeloid cells, such as monocytes, lipidoid formulations may have a similar component molar ratio. Different ratios of lipidoids and other components including, but not limited to, disteroylphosphatidyl choline, cholesterol and PEG-DMG, may be used to optimize the formulation of the polynucleotide for delivery to different cell types including, but not limited to, hepatocytes, myeloid cells, muscle cells, etc. For example, the component molar ratio may include, but is not limited to, 50% C12-200, 10% disteroylphosphatidyl choline, 38.5% cholesterol, and %1.5 PEG-DMG (see Leuschner et al, Nat Biotechnol 2011 29:1005-1010). The use of lipidoid formulations for the localized delivery of nucleic acids to cells (such as, but not limited to, adipose cells and muscle cells) via either subcutaneous or intramuscular delivery, may not require all of the formulation components desired for systemic delivery, and as such may include only the lipidoid and the polynucleotide.

Combinations of different lipidoids may be used to improve the efficacy of polynucleotides directed protein production as the lipidoids may be able to increase cell transfection by the polynucleotide; and/or increase the translation of encoded protein (see Whitehead et al., Mol. Ther. 2011, 19:1688-1694).

In one embodiment, the lipidoid may be prepared from the conjugate addition of alkylamines to acrylates. As a non-limiting example, the lipidoid may be prepared by the methods described in International Patent Publication No. WO2014028487. In one embodiment, the lipidoid may include a compound having formula (I), formula (II), formula (III), formula (IV) or formula (V) as described in International Patent Publication No. WO2014028487. As another non-limiting example, the lipidoid may be prepared using the combinatorial synthesis described by Gu et al. (Biomaterials 2013; 34:6133-6138) such as, but not limited to, the high throughput surface modification methods (HTP). The lipidoids may include at least one of the vinyl amide monomers which were synthesized by the amidation of methacryloyl chloride with amines and grafted onto commercial poly(ether sulfone) (PES) membranes using irradiation from atmospheric pressure plasma (APP). As yet another non-limiting example, the lipidoids described herein may include at least one vinyl amid monomer such as, but not limited to, hydroxyl amide monomers N-(3-hydroxypropyl)methacrylamide (A3), N-(4-hydroxybutyl)methacrylamide (A4), and N-(4-hydroxybutyl) methacrylamide (A6), ethylene glycol (EG) monomer N-(3-methoxypropyl)methacrylamide (A7), and N-(2-(dimethylamino)ethyl)-N-methylmethacrylamide (A8), and N-(2-(diethylamino)ethyl)-N-methylmethacrylamide (A9) all terminated with tertiary amines (see e.g., Gu et al. Biomaterials 2013; 34:6133-6138).

In one embodiment, the lipidoid may be biodegradable.

In one embodiment, the lipid nanoparticles may include polyethylene glycol (PEG), polyethylenimine (PEI), dithiobis(succinimidylpropionate) (DSP), dimethyl-3,3′-dithiobispropionimidate (DTBP), poly(ethylene imine) biscarbamate (PEIC), poly(L-lysine) (PLL), histidine modified PLL, poly(N-vinylpyrrolidone) (PVP), poly(propylenimine) (PPI), poly(amidoamine) (PAMAM), poly(amido ethylenimine) (SS-PAEI), triehtylenetetramine (TETA), poly(β-aminoester), poly(4-hydroxy-L-proine ester) (PHP), poly(allylamine), poly(α-[4-aminobutyl]-L-glycolic acid) (PAGA), poly(D,L-lactic-co-glycolic acid) (PLGA), poly(N-ethyl-4-vinylpyridinium bromide), poly(phosphazene) (PPZ), poly(phosphoester) (PPE), poly(phosphoramidate) (PPA), poly(N-2-hydroxypropylmethacrylamide) (pHPMA), poly(2-(dimethylamino)ethyl methacrylate) (pDMAEMA), poly(2-aminoethyl propylene phosphate) PPE-EA), chitosan, galactosylated chitosan, N-dodecylated chitosan, histone, collagen, and dextran-spermine. In one embodiment, the polymer may be an inert polymer such as, but not limited to, PEG. In one embodiment, the polymer may be a cationic polymer such as, but not limited to, PEI, PLL, TETA, poly(allylamine), poly(N-ethyl-4-vinylpyridinium bromide), pHPMA and pDMAEMA. In one embodiment, the polymer may be a biodegradable PEI such as, but not limited to, DSP, DTBP and PEIC. In one embodiment, the polymer may be biodegradable such as, but not limited to, histidine modified PLL, SS-PAEI, poly(β-aminoester), PHP, PAGA, PLGA, PPZ, PPE, PPA, or PPE-EA.

In one embodiment, the lipid nanoparticles described herein may include a PEG lipid which is a non-diffusible PEG. Non-limiting examples of non-diffusible PEGs include PEG-DSG and PEG-DSPE. As a non-limiting example, the lipid nanoparticle including the PEG lipid includes 40-60% lipid (e.g., DODMA, DLin-KC2-DMA or DLin-MC3-DMA), 8-15% non-cationic lipid of neutral overall charge (e.g., DSPC or DOPE), 30-45% cholesterol, and 0.5-10% PEG lipid (e.g., PEG-DSG or PEG-DSPE). As another non-limiting example, the lipid nanoparticle including the PEG lipid includes 50% lipid (e.g., DODMA, DLin-KC2-DMA or DLin-MC3-DMA), 10% non-cationic lipid of neutral overall charge (e.g., DSPC or DOPE), 39.5%, 38.5%, 35% or 30% cholesterol, and 0.5%, 1.5%, 5% or 10% PEG lipid (e.g., PEG-DSG, PEG-DMG, PEG-DOMG, or PEG-DSPE).

In one embodiment, the lipid nanoparticles may contain PEG-DMG 2000 (1,2-dimyristoyl-sn-glycero-3-phophoethanolamine-N-[methoxy(polyethylene glycol)-2000). In one embodiment, the lipid nanoparticles may contain PEG-DMG 2000, a cationic lipid known in the art and at least one other component. In another embodiment, the lipid nanoparticle formulation may contain PEG-DMG 2000, a cationic lipid known in the art, DSPC and cholesterol. As a non-limiting example, the lipid nanoparticles may contain PEG-DMG 2000, DLin-DMA, DSPC and cholesterol. As another non-limiting example the lipid nanoparticle formulation may contain PEG-DMG 2000, DLin-DMA, DSPC and cholesterol in a molar ratio of 2:40:10:48 (see e.g., Geall et al., Proc Natl Acad Sci U.S.A. 2012; PMID: 22908294).

The lipid nanoparticles may include a phosphate conjugate. The phosphate conjugate may increase in vivo circulation times and/or increase the targeted delivery of the nanoparticle. Phosphate conjugates for use with the present invention may be made by the methods described in International Patent Publication No. WO2013033438 or U.S. Publication No. 20130196948. As a non-limiting example, the phosphate conjugates may include a compound of any one of the formulas described in International Patent Publication No. WO2013033438.

The nanoparticles may include a polymer conjugate. The polymer conjugate may be a water soluble conjugate. The polymer conjugate may have a structure as described in U.S. Publication No. 20130059360. In one aspect, polymer conjugates with the polynucleotides of the present invention may be made using the methods and/or segmented polymeric reagents described in U.S. Publication No. 20130072709. In another aspect, the polymer conjugate may have pendant side groups including ring moieties such as, but not limited to, the polymer conjugates described in U.S. Publication No. 20130196948.

In one embodiment, the polynucleotides of the invention may be part of a nucleic acid conjugate including a hydrophobic polymer covalently bound to the polynucleotide through a first linker wherein said conjugate forms nanoparticulate micelles having a hydrophobic core and a hydrophilic shell, for example, to render nucleic acids resistant to nuclease digestion, as described in International Patent Publication No. WO2014047649.

The nanoparticles may include a conjugate to enhance the delivery of nanoparticles of the present invention in a subject. Further, the conjugate may inhibit phagocytic clearance of the nanoparticles in a subject. In one aspect, the conjugate may be a “self” peptide designed from the human membrane protein CD47 (e.g., the “self” particles described by Rodriguez et al (Science 2013 339, 971-975)). As shown by Rodriguez et al. the self peptides delayed macrophage-mediated clearance of nanoparticles which enhanced delivery of the nanoparticles. In another aspect, the conjugate may be the membrane protein CD47 (e.g., see Rodriguez et al. Science 2013 339, 971-975). Rodriguez et al. showed that, similarly to “self” peptides, CD47 can increase the circulating particle ratio in a subject as compared to scrambled peptides and PEG coated nanoparticles.

In one embodiment, the nanoparticles include a conjugate to enhance the delivery of the nanoparticles of the present invention in a subject. The conjugate may be the CD47 membrane or the conjugate may be derived from the CD47 membrane protein, such as the “self” peptide described previously. In another aspect the nanoparticle may include PEG and a conjugate of CD47 or a derivative thereof. In yet another aspect, the nanoparticle may include both the “self” peptide described above and the membrane protein CD47.

In another aspect, a “self” peptide and/or CD47 protein may be conjugated to a virus-like particle or pseudovirion, as described herein for delivery of the polynucleotides of the present invention.

In another embodiment, HIF-1 inhibitors may be conjugated to or dispersed in controlled release formulations such as a polymer-conjugate as described in International Patent Publication No. WO2013138343. The polynucleotides described herein may encode HIF-1 inhibitors and may be delivered using the controlled release formulations of polymer-conjugates. The polymer-conjugates including HIF-1 inhibitors may be used to treat a disease and/or disorder that is associated with vascularization such as, but not limited to, cancer, obesity, and ocular diseases such as wet age-related macular degeneration (AMD).

In another embodiment, the lipid nanoparticle may include a conjugation of at least one targeting ligand. The targeting ligand may be any ligand known in the art such as, but not limited to, a monoclonal antibody (Kirpotin et al, Cancer Res. 2006 66:6732-6740).

In one embodiment, the particle includes a conjugate for delivering nucleic acid agents such as the particles described in U.S. Publication No. US20140037573. As a non-limiting example, the particle may include a plurality of hydrophobic moieties, or a plurality of hydrophilic-hydrophobic polymers and nucleic acid agents.

In one embodiment, albumin-binding lipids may be conjugated to cargo (e.g., the polynucleotides and formulations thereof) for targeted delivery to the lymph nodes. Non-limiting examples of albumin-binding lipids and conjugates thereof are described in International Patent Publication No. WO2013151771.

Lipid nanoparticles of the present invention may be coated with a surfactant or polymer in order to improve the delivery of the particle. In one embodiment, the nanoparticle may be coated with a hydrophilic coating such as, but not limited to, PEG coatings and/or coatings that have a neutral surface charge. The hydrophilic coatings may help to deliver nanoparticles with larger payloads such as, but not limited to, polynucleotides within the central nervous system. As a non-limiting example nanoparticles including a hydrophilic coating and methods of making such nanoparticles are described in U.S. Publication No. US20130183244.

Lipid nanoparticles may be engineered to alter the surface properties of particles so the lipid nanoparticles may penetrate the mucosal barrier. Mucus is located on mucosal tissue such as, but not limited to, oral (e.g., the buccal and esophageal membranes and tonsil tissue), ophthalmic, gastrointestinal (e.g., stomach, small intestine, large intestine, colon, rectum), nasal, respiratory (e.g., nasal, pharyngeal, tracheal and bronchial membranes), and genital (e.g., vaginal, cervical, and urethral membranes) tissues. Nanoparticles larger than 10-200 nm, which are preferred for higher drug encapsulation efficiency and the ability to provide the sustained delivery of a wide array of drugs, have been thought to be too large to rapidly diffuse through mucosal barriers. Mucus is continuously secreted, shed, discarded or digested and recycled so most of the trapped particles may be removed from the mucosal tissue within seconds or within a few hours. Large polymeric nanoparticles (200-500 nm in diameter), which have been coated densely with a low molecular weight polyethylene glycol (PEG), diffused through mucus only 4 to 6-fold slower than the same particles diffusing in water (Lai et al. Proc Natl Acad Sci 2007 104(5):1482-487; Lai et al. Adv Drug Deliv Rev. 2009 61(2): 158-171). The transport of nanoparticles may be determined using rates of permeation and/or fluorescent microscopy techniques including, but not limited to, fluorescence recovery after photobleaching (FRAP) and high resolution multiple particle tracking (MPT). As a non-limiting example, compositions which can penetrate a mucosal barrier may be made as described in U.S. Pat. No. 8,241,670 or International Patent Publication No. WO2013110028.

Lipid nanoparticles can be engineered to penetrate mucus may include a polymeric material (i.e. a polymeric core) and/or a polymer-vitamin conjugate and/or a tri-block co-polymer. The polymeric material may include, but is not limited to, polyamines, polyethers, polyamides, polyesters, polycarbamates, polyureas, polycarbonates, poly(styrenes), polyimides, polysulfones, polyurethanes, polyanhydrides, polyacetylenes, polyethylenes, polyethyeneimines, polyisocyanates, polyacrylates, polymethacrylates, polyacrylonitriles, and polyarylates. The polymeric material may be biodegradable and/or biocompatible. Non-limiting examples of biocompatible polymers are described in International Patent Publication No. WO2013116804. The polymeric material may additionally be irradiated. As a non-limiting example, the polymeric material may be gamma irradiated (see e.g., International Patent Publication No. WO201282165). Non-limiting examples of specific polymers include poly(caprolactone) (PCL), ethylene vinyl acetate polymer (EVA), poly(lactic acid) (PLA), poly(L-lactic acid) (PLLA), poly(glycolic acid) (PGA), poly(lactic acid-co-glycolic acid) (PLGA), poly(L-lactic acid-co-glycolic acid) (PLLGA), poly(D,L-lactide) (PDLA), poly(L-lactide) (PLLA), poly(D,L-lactide-co-caprolactone), poly(D,L-lactide-co-caprolactone-co-glycolide), poly(D,L-lactide-co-PEO-co-D,L-lactide), poly(D,L-lactide-co-PPO-co-D,L-lactide), polyalkyl cyanoacralate, polyurethane, poly-L-lysine (PLL), hydroxypropyl methacrylate (HPMA), polyethyleneglycol, poly-L-glutamic acid, poly(hydroxy acids), polyanhydrides, polyorthoesters, poly(ester amides), polyamides, poly(ester ethers), polycarbonates, polyalkylenes such as polyethylene and polypropylene, polyalkylene glycols such as poly(ethylene glycol) (PEG), polyalkylene oxides (PEO), polyalkylene terephthalates such as poly(ethylene terephthalate), polyvinyl alcohols (PVA), polyvinyl ethers, polyvinyl esters such as poly(vinyl acetate), polyvinyl halides such as poly(vinyl chloride) (PVC), polyvinylpyrrolidone, polysiloxanes, polystyrene (PS), polyurethanes, derivatized celluloses such as alkyl celluloses, hydroxyalkyl celluloses, cellulose ethers, cellulose esters, nitro celluloses, hydroxypropylcellulose, carboxymethylcellulose, polymers of acrylic acids, such as poly(methyl(meth)acrylate) (PMMA), poly(ethyl(meth)acrylate), poly(butyl(meth)acrylate), poly(isobutyl(meth)acrylate), poly(hexyl(meth)acrylate), poly(isodecyl(meth)acrylate), poly(lauryl(meth)acrylate), poly(phenyl(meth)acrylate), poly(methyl acrylate), poly(isopropyl acrylate), poly(isobutyl acrylate), poly(octadecyl acrylate) and copolymers and mixtures thereof, polydioxanone and its copolymers, polyhydroxyalkanoates, polypropylene fumarate, polyoxymethylene, poloxamers, poly(ortho)esters, poly(butyric acid), poly(valeric acid), poly(lactide-co-caprolactone), PEG-PLGA-PEG, trimethylene carbonate, or polyvinylpyrrolidone. The lipid nanoparticle may be coated or associated with a co-polymer such as, but not limited to, a block co-polymer (such as a branched polyether-polyamide block copolymer described in International Patent Publication No. WO2013012476 and EP2734184), and (poly(ethylene glycol))-(poly(propylene oxide))-(poly(ethylene glycol)) triblock copolymer (see e.g., U.S. Publication No. 20120121718, U.S. Publication No. 20100003337, and U.S. Pat. No. 8,263,665). The co-polymer may be a polymer that is generally regarded as safe (GRAS) and the formation of the lipid nanoparticle may be in such a way that no new chemical entities are created. For example, the lipid nanoparticle may include poloxamers coating PLGA nanoparticles without forming new chemical entities which are still able to rapidly penetrate human mucus (Yang et al. Angew. Chem. Int. Ed. 2011 50:2597-2600). A non-limiting scalable method to produce nanoparticles which can penetrate human mucus is described by Xu et al. (See e.g., J Control Release 2013, 170(2):279-86).

The lipid nanoparticle engineered to penetrate mucus may include surface altering agents such as, but not limited to, polynucleotides, anionic proteins (e.g., bovine serum albumin), surfactants (e.g., cationic surfactants such as for example dimethyldioctadecyl-ammonium bromide), sugars or sugar derivatives (e.g., cyclodextrin), nucleic acids, polymers (e.g., heparin, polyethylene glycol and poloxamer), mucolytic agents (e.g., N-acetylcysteine, mugwort, bromelain, papain, clerodendrum, acetylcysteine, bromhexine, carbocisteine, eprazinone, mesna, ambroxol, sobrerol, domiodol, letosteine, stepronin, tiopronin, gelsolin, thymosin P4 dornase alfa, neltenexine, erdosteine), and various DNases including rhDNase. The surface altering agent may be embedded or enmeshed in the particle's surface or disposed (e.g., by coating, adsorption, covalent linkage, or other process) on the surface of the lipid nanoparticle. (see, e.g., U.S. Publication Nos. 20100215580, 20080166414, and 20130164343).

In one embodiment, the mucus penetrating lipid nanoparticles may include at least one polynucleotide described herein. The polynucleotide may be encapsulated in the lipid nanoparticle and/or disposed on the surface of the nanoparticle. The polynucleotide may be covalently coupled to the lipid nanoparticle. Formulations of mucus penetrating lipid nanoparticles may include a plurality of nanoparticles. Further, the formulations may contain particles which may interact with the mucus and alter the structural and/or adhesive properties of the surrounding mucus to decrease mucoadhesion which may increase the delivery of the mucus penetrating lipid nanoparticles to the mucosal tissue.

In another embodiment, the mucus penetrating lipid nanoparticles may be a hypotonic formulation including a mucosal penetration enhancing coating. The formulation may be hypotonic for the epithelium to which it is being delivered. Non-limiting examples of hypotonic formulations may be found in International Patent Publication No. WO2013110028.

In one embodiment, in order to reduce the mucoadhesive properties of a nanoparticle described herein, the nanoparticle may be coated with and/or associated with a triblock copolymer as described in US Patent Publication No. US20130236556 and International Patent Publication No. WO2013166385. As a non-limiting example, the triblock copolymer may be a poly(ethylene glycol)-poly(propylene oxide)-poly(ethylene glycol) triblock copolymer as described in U.S. Publication No. 20130236556 and International Patent Publication No. WO2013166385. As another non-limiting example, the nanoparticle with reduced mucoadhesive may be prepared by the methods described in U.S. Publication No. 20130236556 and International Patent Publication No. WO2013166385.

In one embodiment, mucus-penetrating particles (MPP) without any or with minimal use of polymeric carriers may be used to deliver and/or formulate the polynucleotides described herein. As a non-limiting example, the MPP may be a nanocrystal or a particle as described in U.S. Publication No. 20130323179.

In one embodiment, the lipid nanoparticles are mucoadhesive system. As a non-limiting example, the mucoadhesive nanoparticle are described in International Patent Publication No. WO2013188979. The nanoparticles may include a plurality of amphiphilic macromolecules which may contain a hydrophobic portion, a hydrophilic portion having multiple functional moieties and a mucosal targeting moiety.

In one embodiment, the lipid nanoparticles are mucus-penetrating liposomal nanoparticles described in International Patent Publication No. WO2013166498. The nanoparticles may contain therapeutic agents to be delivered to a mucosal surface, one or more lipids, one or more PEG-conjugated lipids and one or more additional materials to physically and/or chemically stabilize the particles.

In one embodiment, the lipid nanoparticles are neurophilic nanoparticles. Neurophilic nanoparticles may be useful to deliver compounds (e.g., compounds suitable for therapeutic purposes) to cells found in the peripheral nervous system and/or endothelial cells that form the blood brain barrier. The neurophilic nanoparticles may include at least a phospholipid, a non-ionic surfactant and a cholesterol. As a non-limiting example, the neurophilic nanoparticles are the liposomal nanoparticles described in International Patent Publication No. WO2013151650. These neurophilic nanoparticles may be advantageous for targeting neural cells, endothelial cells of the blood vessels and epithelial cells of the choroid plexus that serve the brain.

In one embodiment, the lipid nanoparticle is a lipoplex, such as, without limitation, the ATUPLEX™ system, the DACC system, the DBTC system, or another siRNA-lipoplex technology, e.g., from Silence Therapeutics (London, United Kingdom), STEMFECT™ from STEMGENT® (Cambridge, Mass.), and polyethylenimine (PEI) or protamine-based targeted and non-targeted delivery of nucleic acids acids (Aleku et al. Cancer Res. 2008 68:9788-9798; Strumberg et al. Int J Clin Pharmacol Ther 2012 50:76-78; Santel et al., Gene Ther 2006 13:1222-1234; Santel et al., Gene Ther 2006 13:1360-1370; Gutbier et al., Pulm Pharmacol. Ther. 2010 23:334-344; Kaufmann et al. Microvasc Res 2010 80:286-293; Weide et al. J Immunother. 2009 32:498-507; Weide et al. J Immunother. 2008 31:180-188; Pascolo Expert Opin. Biol. Ther. 4:1285-1294; Fotin-Mleczek et al., 2011 J. Immunother. 34:1-15; Song et al., Nature Biotechnol. 2005, 23:709-717; Peer et al., Proc Natl Acad Sci USA. 2007 6; 104:4095-4100; deFougerolles Hum Gene Ther. 2008 19:125-132).

In one embodiment, such formulations may also be constructed or compositions altered such that they passively or actively are directed to different cell types in vivo, including but not limited to hepatocytes, immune cells, tumor cells, endothelial cells, antigen presenting cells, and leukocytes (Akinc et al. Mol Ther. 2010 18:1357-1364; Song et al., Nat Biotechnol. 2005 23:709-717; Judge et al., J Clin Invest. 2009 119:661-673; Kaufmann et al., Microvasc Res 2010 80:286-293; Santel et al., Gene Ther 2006 13:1222-1234; Santel et al., Gene Ther 2006 13:1360-1370; Gutbier et al., Pulm Pharmacol. Ther. 2010 23:334-344; Basha et al., Mol. Ther. 2011 19:2186-2200; Fenske and Cullis, Expert Opin Drug Deliv. 2008 5:25-44; Peer et al., Science. 2008 319:627-630; Peer and Lieberman, Gene Ther. 2011 18:1127-1133). One example of passive targeting of formulations to liver cells includes the DLin-DMA, DLin-KC2-DMA and DLin-MC3-DMA-based lipid nanoparticle formulations which have been shown to bind to apolipoprotein E and promote binding and uptake of these formulations into hepatocytes in vivo (Akinc et al. Mol Ther. 2010 18:1357-1364). Formulations can also be selectively targeted through expression of different ligands on their surface as exemplified by, but not limited by, folate, transferrin, N-acetylgalactosamine (GalNAc), and antibody targeted approaches (Kolhatkar et al., Curr Drug Discov Technol. 2011 8:197-206; Musacchio and Torchilin, Front Biosci. 2011 16:1388-1412; Yu et al., Mol Membr Biol. 2010 27:286-298; Patil et al., Crit Rev Ther Drug Carrier Syst. 2008 25:1-61; Benoit et al., Biomacromolecules. 2011 12:2708-2714; Zhao et al., Expert Opin Drug Deliv. 2008 5:309-319; Akinc et al., Mol Ther. 2010 18:1357-1364; Srinivasan et al., Methods Mol Biol. 2012 820:105-116; Ben-Arie et al., Methods Mol Biol. 2012 757:497-507; Peer 2010 J Control Release. 20:63-68; Peer et al., Proc Natl Acad Sci USA. 2007 104:4095-4100; Kim et al., Methods Mol Biol. 2011 721:339-353; Subramanya et al., Mol Ther. 2010 18:2028-2037; Song et al., Nat Biotechnol. 2005 23:709-717; Peer et al., Science. 2008 319:627-630; Peer and Lieberman, Gene Ther. 2011 18:1127-1133).

In yet another non-limiting example, the lipid nanoparticle includes the block copolymer PEG-PLGA-PEG (see e.g., the thermosensitive hydrogel (PEG-PLGA-PEG) was used as a TGF-beta1 gene delivery vehicle in Lee et al. (Pharmaceutical Research. 2003 20(12): 1995-2000); as a controlled gene delivery system in Li et al. (Pharmaceutical Research. 2003 20(6):884-888) and Chang et al. (J. Controlled Release. 2007 118:245-253). The polynucleotides of the present invention may be formulated in lipid nanoparticles including the PEG-PLGA-PEG block copolymer.

In one embodiment, the average or mean size of the nanoparticles in the nanoparticle formulation may be from about 10 to about 100 nm such as, but not limited to, about 10 to about 20 nm, about 10 to about 30 nm, about 10 to about 40 nm, about 10 to about 50 nm, about 10 to about 60 nm, about 10 to about 70 nm, about 10 to about 80 nm, about 10 to about 90 nm, about 20 to about 30 nm, about 20 to about 40 nm, about 20 to about 50 nm, about 20 to about 60 nm, about 20 to about 70 nm, about 20 to about 80 nm, about 20 to about 90 nm, about 20 to about 100 nm, about 30 to about 40 nm, about 30 to about 50 nm, about 30 to about 60 nm, about 30 to about 70 nm, about 30 to about 80 nm, about 30 to about 90 nm, about 30 to about 100 nm, about 40 to about 50 nm, about 40 to about 60 nm, about 40 to about 70 nm, about 40 to about 80 nm, about 40 to about 90 nm, about 40 to about 100 nm, about 50 to about 60 nm, about 50 to about 70 nm, about 50 to about 80 nm, about 50 to about 90 nm, about 50 to about 100 nm, about 60 to about 70 nm, about 60 to about 80 nm, about 60 to about 90 nm, about 60 to about 100 nm, about 60 to about 225 nm, about 70 to about 80 nm, about 70 to about 90 nm, about 70 to about 100 nm, about 80 to about 90 nm, about 80 to about 100 nm and/or about 90 to about 100 nm.

In other embodiments, the lipid nanoparticles may have a diameter from about 80 to 100 nm, 85 to 100 nm, 90 to 100 nm, 95 to 100 nm, 80 to 95 nm, 80 to 90 nm, 80 to 85 nm, 85 to 90 nm, 85 to 95 nm, or 90 to 95 nm. In other embodiments, the lipid nanoparticles may have a diameter from about 90 to 110 nm, 95 to 110 nm, 100 to 110 nm, 105 to 110 nm, 90 to 105 nm, 90 to 100 nm, 90 to 95 nm, 95 to 105 nm, 95 to 100 nm, or 110 to 105 nm. In other embodiments, the lipid nanoparticles may have a diameter from about 100 to 120 nm, 100 to 115 nm, 100 to 110 nm, 100 to 105 nm, 105 to 120 nm, 105 to 115 nm, 105 to 110 nm, 110 to 115 nm, 110 to 120 nm, or 115 to 120 nm.

In one embodiment, the lipid nanoparticles may have a diameter from about 10 to 500 nm. This diameter may be the mean or average size of the nanoparticles in a nanoparticle formulation.

In one embodiment, the lipid nanoparticle may have a diameter greater than 100 nm, greater than 150 nm, greater than 200 nm, greater than 250 nm, greater than 300 nm, greater than 350 nm, greater than 400 nm, greater than 450 nm, greater than 500 nm, greater than 550 nm, greater than 600 nm, greater than 650 nm, greater than 700 nm, greater than 750 nm, greater than 800 nm, greater than 850 nm, greater than 900 nm, greater than 950 nm, or greater than 1000 nm.

In one embodiment, the average or mean size of the nanoparticles in the nanoparticle formulation may be greater than 100 nm, greater than 150 nm, greater than 200 nm, greater than 250 nm, greater than 300 nm, greater than 350 nm, greater than 400 nm, greater than 450 nm, greater than 500 nm, greater than 550 nm, greater than 600 nm, greater than 650 nm, greater than 700 nm, greater than 750 nm, greater than 800 nm, greater than 850 nm, greater than 900 nm, greater than 950 nm or greater than 1000 nm. In one embodiment, the therapeutic nanoparticles may have a diameter of about 70 to about 130 nm.

In one aspect, the lipid nanoparticle may be a limit size lipid nanoparticle described in International Patent Publication No. WO2013059922. The limit size lipid nanoparticle may include a lipid bilayer surrounding an aqueous core or a hydrophobic core; where the lipid bilayer may include a phospholipid such as, but not limited to, diacylphosphatidylcholine, a diacylphosphatidylethanolamine, a ceramide, a sphingomyelin, a dihydrosphingomyelin, a cephalin, a cerebroside, a C8-C20 fatty acid diacylphophatidylcholine, and 1-palmitoyl-2-oleoyl phosphatidylcholine (POPC). In another aspect the limit size lipid nanoparticle may include a polyethylene glycol-lipid such as, but not limited to, DLPE-PEG, DMPE-PEG, DPPC-PEG and DSPE-PEG.

As a non-limiting example, the lipid nanoparticles described herein may include mRNA in a lipid:mRNA weight ratio of 20:1. As another non-limiting example, the lipid nanoparticle includes 40-60% lipid (e.g., DODMA, DLin-KC2-DMA or DLin-MC3-DMA), 8-15% non-cationic lipid of neutral overall charge (e.g., DSPC or DOPE), 30-45% cholesterol and 1-5% PEG lipid (e.g., PEG 2000-DMG or anionic mPEG-DSPC). As yet another non-limiting example, the lipid nanoparticle includes 50% lipid (e.g., DODMA, DLin-KC2-DMA or DLin-MC3-DMA), 10% non-cationic lipid of neutral overall charge (e.g., DSPC or DOPE), 38.5% cholesterol and 1.5% PEG lipid (e.g., PEG 2000-DMG).

In one embodiment, formulations including the polynucleotides and lipid nanoparticles described herein may include 0.15 mg/ml to 2 mg/ml of the polynucleotide described herein (e.g., mRNA), 50% lipid (e.g., DLin-MC3-DMA), 38.5% Cholesterol, 10% non-cationic lipid of neutral overall charge (e.g., DSPC), 1.5% PEG lipid (e.g., PEG-2K-DMG), 10 mM of citrate buffer, and the formulation may additionally include up to 10% w/w of sucrose (e.g., at least 1% w/w, at least 2% w/w, at least 3% w/w, at least 4% w/w, at least 5% w/w, at least 6% w/w, at least 7% w/w, at least 8% w/w, at least 9% w/w or 10% w/w).

In one embodiment, the lipid nanoparticle is an active substance release system (see, e.g., U.S. Publication No. 20130102545). The active substance release system may include 1) at least one nanoparticle bonded to an oligonucleotide inhibitor strand which is hybridized with a catalytically active nucleic acid and 2) a compound bonded to at least one substrate molecule bonded to a therapeutically active substance (e.g., polynucleotides described herein), where the therapeutically active substance is released by the cleavage of the substrate molecule by the catalytically active nucleic acid.

In another embodiment, the lipid nanoparticles are suitable for use in imaging. The nanoparticles may be liposome nanoparticles such as those described in U.S. Publication No. 20130129636. As a non-limiting example, the liposome may include gadolinium(III)2-{4,7-bis-carboxymethyl-10-[(N,N-distearylamidomethyl-N′-amido-methyl]-1,4,7,10-tetra-azacyclododec-1- yl}-acetic acid and a neutral, fully saturated phospholipid component (see e.g., US Patent Publication No US20130129636).

The lipid nanoparticles of the present invention may further include nutrients such as, but not limited to, those which deficiencies can lead to health hazards from anemia to neural tube defects (see, e.g, the nanoparticles described in International Patent Publication No. WO2013072929). As a non-limiting example, the nutrient may be iron in the form of ferrous, ferric salts or elemental iron, iodine, folic acid, vitamins or micronutrients.

### Apparatus

The present invention provides apparatus for producing lipid nanoparticles, e.g., according to any of the preceding methods. An apparatus of the invention includes an injector (e.g., a pipettor) configured to transfer a lower alkanol solution from a first reservoir to a second reservoir configured to hold an aqueous solution and a pump (e.g., servo pump) configured to operate the injector. In some embodiments, apparatus are configured to repeat the injection multiple times for high throughput processing of lipid nanoparticles.

An apparatus of the invention can include one or more elements of a conventional liquid handling robot. A liquid handling robot operates above a deck that can be integrally connected to (e.g., serve as the base of) the apparatus, upon which one or more reservoirs can be positioned. The deck can alternatively be a physical assembly of smaller units (e.g., blocks) that fit together to form a larger operating surface. A liquid handling robot generally includes an injector, which is supported by an arm. The arm can be attached to a body (e.g., at a slidable connection) to allow displacement of the injector/arm complex along, e.g., an X and/or a Y axis. A liquid handling robot body can include additional hardware, e.g., a power chord connector, a tip waste tray, or a pipette rack. The arm constitutes the main electromechanical element: it generates movement of the injector in space, moving over a two dimensional surface (i.e., along an X-Y plane) but also capable of lifting and descending (along a Z axis) the injector in order to perform the desired injector action. Additionally, the arm may include a camera or other optical sensor, the functionality of manipulating the injector for the purpose of aspiration and dispensing, the functionality of tip ejection and the functionality of actuating the pipette for the purpose of setting a desired volume. Liquid handling robots are described, e.g., in U.S. Pat. No. 7,390,460.

One or more functions of a liquid handling robot can be controlled through a software interface. In some embodiments, a program (e.g., a python script) allows the user to enter parameters, such as, but not limited to, target lipid nanoparticle size, starting injector location, starting reservoir location, final injector location, final reservoir location, flow rate, frequency of injection, injection volumes, and file properties. The software may create a script (e.g., TCS XYZ Pump Commander) executable by one or more elements of the apparatus according to user input.

The first reservoir contains the lower alkanol solution, which can be positioned anywhere on or in the proximity of the apparatus (e.g., on a deck, body, or arm or the apparatus) and connected to the injector by tubing. In one embodiment, the first reservoir is positioned at or near the injector. Positioning the first reservoir at or near the injector minimizes the length of tubing required to connect the lower alkanol solution to the injector. This close proximity can enhance precision by minimizing vacant space within the tubing and reduce the unusable fraction of lower alkanol solution left over in the tube.

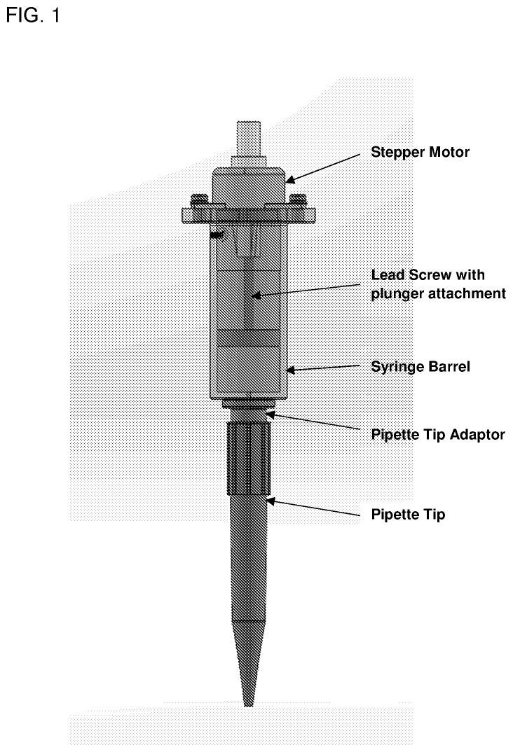

In some embodiments, a stepper motor moves a plunger inside the tube, allowing liquid to be pulled in or pushed out, as shown in FIG. 1. Servo-pumps allow for very high precision of flow and volume at a high flow rate. Precise flow volumes and/or high flow rates allows for small, consistent particle synthesis.

The second reservoir containing the aqueous solution can be placed on the deck, such that the injector can move relative to the second reservoir.

Injectors can be configured to automatically attach and/or remove tips (e.g., pipette tips) between injections. Accordingly, apparatus of the invention can include tip holders that are accessible to the injector during automatic, repeating injection protocols.

The apparatus of the invention can include multiple injectors positioned on the same or different arms. Additionally or alternatively, the one or more injectors can each include one or more channels, e.g., to each inject the lower alkanol solution into the same or separate aqueous solution reservoirs. Injectors having multiple channels (e.g., arranged in one or more rows) are known in the art.

### Formulations

The present invention provides lipid nanoparticles, with or without an encapsulated nucleic acid (such as an alternative nucleic acid), in combination with one or more pharmaceutically acceptable excipients. Pharmaceutical compositions may optionally include one or more additional active substances, e.g. therapeutically and/or prophylactically active substances. Pharmaceutical compositions of the present invention may be sterile and/or pyrogen-free. General considerations in the formulation and/or manufacture of pharmaceutical agents may be found, for example, in Remington (The Science and Practice of Pharmacy, 22nd Edition, A. R. Gennaro, Lippincott, Williams & Wilkins, Baltimore, Md., 2012).

In some embodiments, a pharmaceutically acceptable excipient is at least 95%, at least 96%, at least 97%, at least 98%, at least 99%, or 100% pure. In some embodiments, an excipient is approved for use in humans and for veterinary use. In some embodiments, an excipient is approved by United States Food and Drug Administration. In some embodiments, an excipient is pharmaceutical grade. In some embodiments, an excipient meets the standards of the United States Pharmacopoeia (USP), the European Pharmacopoeia (EP), the British Pharmacopoeia, and/or the International Pharmacopoeia.

Pharmaceutically acceptable excipients used in the manufacture of pharmaceutical compositions include, but are not limited to, inert diluents, dispersing and/or granulating agents, surface active agents and/or emulsifiers, disintegrating agents, binding agents, preservatives, buffering agents, lubricating agents, and/or oils. Such excipients may optionally be included in pharmaceutical formulations. Excipients such as cocoa butter and suppository waxes, coloring agents, coating agents, sweetening, flavoring, and/or perfuming agents can be present in the composition, according to the judgment of the formulator.

Exemplary diluents include, but are not limited to, calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, etc., and/or combinations thereof.

Exemplary granulating and/or dispersing agents include, but are not limited to, potato starch, corn starch, tapioca starch, sodium starch glycolate, clays, alginic acid, guar gum, citrus pulp, agar, bentonite, cellulose and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (Veegum), sodium lauryl sulfate, quaternary ammonium compounds, etc., and/or combinations thereof.

Exemplary surface active agents and/or emulsifiers include, but are not limited to, natural emulsifiers (e.g. acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g. bentonite [aluminum silicate] and Veegum® [magnesium aluminum silicate]), long chain amino acid derivatives, high molecular weight alcohols (e.g. stearyl alcohol, cetyl alcohol, oleyl alcohol, triacetin monostearate, ethylene glycol distearate, glyceryl monostearate, and propylene glycol monostearate, polyvinyl alcohol), carbomers (e.g. carboxy polymethylene, polyacrylic acid, acrylic acid polymer, and carboxyvinyl polymer), carrageenan, cellulosic derivatives (e.g. carboxymethylcellulose sodium, powdered cellulose, hydroxymethyl cellulose, hydroxypropyl cellulose, hydroxypropyl methylcellulose, methylcellulose), sorbitan fatty acid esters (e.g. polyoxyethylene sorbitan monolaurate [Tween® 20], polyoxyethylene sorbitan [Tween® 60], polyoxyethylene sorbitan monooleate [Tween® 80], sorbitan monopalmitate [Span® 40], sorbitan monostearate [Span® 60], sorbitan tristearate [Span® 65], glyceryl monooleate, sorbitan monooleate [Span® 80]), polyoxyethylene esters (e.g. polyoxyethylene monostearate [Myrj® 45], polyoxyethylene hydrogenated castor oil, polyethoxylated castor oil, polyoxymethylene stearate, and Solutol®), sucrose fatty acid esters, polyethylene glycol fatty acid esters (e.g. Cremophor®), polyoxyethylene ethers, (e.g. polyoxyethylene lauryl ether [Brij® 30]), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic® F 68, Poloxamer® 188, cetrimonium bromide, cetylpyridinium chloride, benzalkonium chloride, docusate sodium, etc., and/or combinations thereof.

Exemplary binding agents include, but are not limited to, starch (e.g. cornstarch and starch paste), gelatin, sugars (e.g., sucrose, glucose, dextrose, dextrin, molasses, lactose, lactitol, or mannitol), natural and synthetic gums (e.g., acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum®), and larch arabogalactan), alginates, polyethylene oxide, polyethylene glycol, inorganic calcium salts, silicic acid, polymethacrylates, waxes, water, alcohol, etc., and combinations thereof.

Exemplary preservatives may include, but are not limited to, antioxidants, chelating agents, antimicrobial preservatives, antifungal preservatives, alcohol preservatives, acidic preservatives, and/or other preservatives. Exemplary antioxidants include, but are not limited to, alpha tocopherol, ascorbic acid, acorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and/or sodium sulfite. Exemplary chelating agents include ethylenediaminetetraacetic acid (EDTA), citric acid monohydrate, disodium edetate, dipotassium edetate, edetic acid, fumaric acid, malic acid, phosphoric acid, sodium edetate, tartaric acid, and/or trisodium edetate. Exemplary antimicrobial preservatives include, but are not limited to, benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and/or thimerosal. Exemplary antifungal preservatives include, but are not limited to, butyl paraben, methyl paraben, ethyl paraben, propyl paraben, benzoic acid, hydroxybenzoic acid, potassium benzoate, potassium sorbate, sodium benzoate, sodium propionate, and/or sorbic acid. Exemplary alcohol preservatives include, but are not limited to, ethanol, polyethylene glycol, phenol, phenolic compounds, bisphenol, chlorobutanol, hydroxybenzoate, and/or phenylethyl alcohol. Exemplary acidic preservatives include, but are not limited to, vitamin A, vitamin C, vitamin E, beta-carotene, citric acid, acetic acid, dehydroacetic acid, ascorbic acid, sorbic acid, and/or phytic acid. Other preservatives include, but are not limited to, tocopherol, tocopherol acetate, deteroxime mesylate, cetrimide, butylated hydroxyanisol (BHA), butylated hydroxytoluened (BHT), ethylenediamine, sodium lauryl sulfate (SLS), sodium lauryl ether sulfate (SLES), sodium bisulfite, sodium metabisulfite, potassium sulfite, potassium metabisulfite, Glydant Plus®, Phenonip®, methylparaben, Germall® 115, Germaben®, Neolone™, Kathon™, and/or Euxyl®.

Exemplary buffering agents include, but are not limited to, citrate buffer solutions, acetate buffer solutions, phosphate buffer solutions, ammonium chloride, calcium carbonate, calcium chloride, calcium citrate, calcium glubionate, calcium gluceptate, calcium gluconate, d-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, etc., and/or combinations thereof.

Exemplary lubricating agents include, but are not limited to, magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, etc., and/or combinations thereof.

Exemplary oils include, but are not limited to, almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and/or combinations thereof.

Liquid dosage forms for oral and parenteral administration include, but are not limited to, pharmaceutically acceptable emulsions, microemulsions, solutions, suspensions, syrups, and/or elixirs. In addition to active ingredients, liquid dosage forms may include inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, oral compositions can include adjuvants such as wetting agents, emulsifying and suspending agents, sweetening, flavoring, and/or perfuming agents. In certain embodiments for parenteral administration, compositions are mixed with solubilizing agents such as Cremophor®, alcohols, oils, modified oils, glycols, polysorbates, cyclodextrins, polymers, and/or combinations thereof.

Injectable preparations, for example, sterile injectable aqueous or oleaginous suspensions may be formulated according to the known art using suitable dispersing agents, wetting agents, and/or suspending agents. Sterile injectable preparations may be sterile injectable solutions, suspensions, and/or emulsions in nontoxic parenterally acceptable diluents and/or solvents, for example, as a solution in 1,3-butanediol. Among the acceptable vehicles and solvents that may be employed are water, Ringer's solution, U.S.P., and isotonic sodium chloride solution. Sterile, fixed oils are conventionally employed as a solvent or suspending medium. For this purpose any bland fixed oil can be employed including synthetic mono- or diglycerides. Fatty acids such as oleic acid can be used in the preparation of injectables.

Injectable formulations can be sterilized, for example, by filtration through a bacterial-retaining filter, and/or by incorporating sterilizing agents in the form of sterile solid compositions which can be dissolved or dispersed in sterile water or other sterile injectable medium prior to use.

In order to prolong the effect of an active ingredient, it is often desirable to slow the absorption of the active ingredient from subcutaneous or intramuscular injection. This may be accomplished by the use of a liquid suspension of crystalline or amorphous material with poor water solubility. The rate of absorption of the drug then depends upon its rate of dissolution which, in turn, may depend upon crystal size and crystalline form. Alternatively, delayed absorption of a parenterally administered drug form is accomplished by dissolving or suspending the drug in an oil vehicle. Injectable depot forms are made by forming microencapsule matrices of the drug in biodegradable polymers such as polylactide-polyglycolide. Depending upon the ratio of drug to polymer and the nature of the particular polymer employed, the rate of drug release can be controlled. Examples of other biodegradable polymers include poly(orthoesters) and poly(anhydrides). Depot injectable formulations are prepared by entrapping the drug in liposomes or microemulsions which are compatible with body tissues.

Compositions for rectal or vaginal administration are typically suppositories which can be prepared by mixing compositions with suitable non-irritating excipients such as cocoa butter, polyethylene glycol or a suppository wax which are solid at ambient temperature but liquid at body temperature and therefore melt in the rectum or vaginal cavity and release the active ingredient.

Solid dosage forms for oral administration include capsules, tablets, pills, powders, and granules. In such solid dosage forms, an active ingredient is mixed with at least one inert, pharmaceutically acceptable excipient such as sodium citrate or dicalcium phosphate and/or fillers or extenders (e.g. starches, lactose, sucrose, glucose, mannitol, and silicic acid), binders (e.g. carboxymethylcellulose, alginates, gelatin, polyvinylpyrrolidinone, sucrose, and acacia), humectants (e.g. glycerol), disintegrating agents (e.g. agar, calcium carbonate, potato or tapioca starch, alginic acid, certain silicates, and sodium carbonate), solution retarding agents (e.g. paraffin), absorption accelerators (e.g. quaternary ammonium compounds), wetting agents (e.g. cetyl alcohol and glycerol monostearate), absorbents (e.g. kaolin and bentonite clay), and lubricants (e.g. talc, calcium stearate, magnesium stearate, solid polyethylene glycols, sodium lauryl sulfate), and mixtures thereof. In the case of capsules, tablets and pills, the dosage form may include buffering agents.

Solid compositions of a similar type may be employed as fillers in soft and hard-filled gelatin capsules using such excipients as lactose or milk sugar as well as high molecular weight polyethylene glycols and the like. Solid dosage forms of tablets, dragees, capsules, pills, and granules can be prepared with coatings and shells such as enteric coatings and other coatings well known in the pharmaceutical formulating art. They may optionally include opacifying agents and can be of a composition that they release the active ingredient(s) only, or preferentially, in a certain part of the intestinal tract, optionally, in a delayed manner. Examples of embedding compositions which can be used include polymeric substances and waxes. Solid compositions of a similar type may be employed as fillers in soft and hard-filled gelatin capsules using such excipients as lactose or milk sugar as well as high molecular weight polyethylene glycols and the like.

Dosage forms for topical and/or transdermal administration of a composition may include ointments, pastes, creams, lotions, gels, powders, solutions, sprays, inhalants and/or patches. Generally, an active ingredient is admixed under sterile conditions with a pharmaceutically acceptable excipient and/or any needed preservatives and/or buffers as may be required. Additionally, the present disclosure contemplates the use of transdermal patches, which often have the added advantage of providing controlled delivery of a compound to the body. Such dosage forms may be prepared, for example, by dissolving and/or dispensing the compound in the proper medium. Alternatively or additionally, rate may be controlled by either providing a rate controlling membrane and/or by dispersing the compound in a polymer matrix and/or gel.

Suitable devices for use in delivering intradermal pharmaceutical compositions described herein include short needle devices such as those described in U.S. Pat. Nos. 4,886,499, 5,190,521, 5,328,483, 5,527,288, 4,270,537, 5,015,235, 5,141,496, and 5,417,662. Intradermal compositions may be administered by devices which limit the effective penetration length of a needle into the skin, such as those described in International Patent Publication No. WO1999034850 and functional equivalents thereof. Jet injection devices which deliver liquid compositions to the dermis via a liquid jet injector and/or via a needle which pierces the stratum corneum and produces a jet which reaches the dermis are suitable. Jet injection devices are described, for example, in U.S. Pat. Nos. 5,480,381, 5,599,302, 5,334,144, 5,993,412, 5,649,912, 5,569,189, 5,704,911, 5,383,851, 5,893,397, 5,466,220, 5,339,163, 5,312,335, 5,503,627, 5,064,413, 5,520,639, 4,596,556, 4,790,824, 4,941,880, 4,940,460, and International Patent Publication Nos. WO1997037705 and WO1997013537. Ballistic powder/particle delivery devices which use compressed gas to accelerate vaccine in powder form through the outer layers of the skin to the dermis are suitable. Alternatively or additionally, conventional syringes may be used in the classical mantoux method of intradermal administration.

Formulations suitable for topical administration include, but are not limited to, liquid and/or semi liquid preparations such as liniments, lotions, oil in water and/or water in oil emulsions such as creams, ointments and/or pastes, and/or solutions and/or suspensions. Topically-administrable formulations may, for example, include from about 1% to about 10% (w/w) active ingredient, although the concentration of active ingredient may be as high as the solubility limit of the active ingredient in the solvent. Formulations for topical administration may further include one or more of the additional ingredients described herein.

A pharmaceutical composition may be prepared, packaged, and/or sold in a formulation suitable for pulmonary administration via the buccal cavity. Such a formulation may include dry particles which include the active ingredient and which have a diameter in the range from about 0.5 nm to about 7 nm or from about 1 nm to about 6 nm. Such compositions are conveniently in the form of dry powders for administration using a device including a dry powder reservoir to which a stream of propellant may be directed to disperse the powder and/or using a self-propelling solvent/powder dispensing container such as a device including the active ingredient dissolved and/or suspended in a low-boiling propellant in a sealed container. Such powders include particles wherein at least 98% of the particles by weight have a diameter greater than 0.5 nm and at least 95% of the particles by number have a diameter less than 7 nm. Alternatively, at least 95% of the particles by weight have a diameter greater than 1 nm and at least 90% of the particles by number have a diameter less than 6 nm. Dry powder compositions may include a solid fine powder diluent such as sugar and are conveniently provided in a unit dose form.

Low boiling propellants generally include liquid propellants having a boiling point of below 65° F. at atmospheric pressure. Generally the propellant may constitute 50% to 99.9% (w/w) of the composition, and active ingredient may constitute 0.1% to 20% (w/w) of the composition. A propellant may further include additional ingredients such as a liquid non-ionic and/or solid anionic surfactant and/or a solid diluent (which may have a particle size of the same order as particles including the active ingredient).

Pharmaceutical compositions formulated for pulmonary delivery may provide an active ingredient in the form of droplets of a solution and/or suspension. Such formulations may be prepared, packaged, and/or sold as aqueous and/or dilute alcoholic solutions and/or suspensions, optionally sterile, including active ingredient, and may conveniently be administered using any nebulization and/or atomization device. Such formulations may further include one or more additional ingredients including, but not limited to, a flavoring agent such as saccharin sodium, a volatile oil, a buffering agent, a surface active agent, and/or a preservative such as methylhydroxybenzoate. Droplets provided by this route of administration may have an average diameter in the range from about 0.1 nm to about 200 nm.

Formulations described herein as being useful for pulmonary delivery are useful for intranasal delivery of a pharmaceutical composition. Another formulation suitable for intranasal administration is a coarse powder including the active ingredient and having an average particle from about 0.2 μm to 500 μm. Such a formulation is administered in the manner in which snuff is taken, i.e. by rapid inhalation through the nasal passage from a container of the powder held close to the nose.

Formulations suitable for nasal administration may, for example, include from about as little as 0.1% (w/w) and as much as 100% (w/w) of active ingredient, and may include one or more of the additional ingredients described herein. A pharmaceutical composition may be prepared, packaged, and/or sold in a formulation suitable for buccal administration. Such formulations may, for example, be in the form of tablets and/or lozenges made using conventional methods, and may, for example, include 0.1% to 20% (w/w) active ingredient, the balance including an orally dissolvable and/or degradable composition and, optionally, one or more of the additional ingredients described herein. Alternately, formulations suitable for buccal administration may include a powder and/or an aerosolized and/or atomized solution and/or suspension including active ingredient. Such powdered, aerosolized, and/or aerosolized formulations, when dispersed, may have an average particle and/or droplet size in the range from about 0.1 nm to about 200 nm, and may further include one or more of any additional ingredients described herein.

A pharmaceutical composition may be prepared, packaged, and/or sold in a formulation suitable for ophthalmic administration. Such formulations may, for example, be in the form of eye drops including, for example, a 0.1/1.0% (w/w) solution and/or suspension of the active ingredient in an aqueous or oily liquid excipient. Such drops may further include buffering agents, salts, and/or one or more other of any additional ingredients described herein. Other opthalmically-administrable formulations which are useful include those which include the active ingredient in microcrystalline form and/or in a liposomal preparation. Ear drops and/or eye drops are contemplated as being within the scope of this present disclosure.

In some embodiments, lipid nanoparticles including a polynucleotide are administered to humans, human patients or subjects. For the purposes of the present disclosure, the phrase “active ingredient” generally refers to the lipid nanoparticles described herein.

Although the descriptions of pharmaceutical compositions provided herein are principally directed to pharmaceutical compositions which are suitable for administration to humans, it will be understood by the skilled artisan that such compositions are generally suitable for administration to any other animal, e.g., to non-human animals, e.g. non-human mammals. Modification of pharmaceutical compositions suitable for administration to humans in order to render the compositions suitable for administration to various animals is well understood, and the ordinarily skilled veterinary pharmacologist can design and/or perform such modification with merely ordinary, if any, experimentation. Subjects to which administration of the pharmaceutical compositions is contemplated include, but are not limited to, humans and/or other primates; mammals, including commercially relevant mammals such as cattle, pigs, horses, sheep, cats, dogs, mice, rats, and/or birds, including commercially relevant birds such as chickens, ducks, geese, and/or turkeys.

Formulations of the pharmaceutical compositions described herein may be prepared by any method known or hereafter developed in the art of pharmacology. In general, such preparatory methods include the step of bringing the active ingredient into association with an excipient and/or one or more other accessory ingredients, and then, if necessary and/or desirable, dividing, shaping and/or packaging the product into a desired single- or multi-dose unit.

Relative amounts of the active ingredient, the pharmaceutically acceptable excipient, and/or any additional ingredients in a pharmaceutical composition in accordance with the invention will vary, depending upon the identity, size, and/or condition of the subject treated and further depending upon the route by which the composition is to be administered. By way of example, the composition may include between 0.1% and 100%, e.g., between 0.5 and 50%, between 1-30%, between 5-80%, at least 80% (w/w) active ingredient.

The polynucleotides of the invention can be formulated using one or more excipients to: (1) increase stability; (2) increase cell transfection; (3) permit the sustained or delayed release (e.g., from a depot formulation of the polynucleotide); (4) alter the biodistribution (e.g., target the polynucleotide to specific tissues or cell types); (5) increase the translation of encoded protein in vivo; and/or (6) alter the release profile of encoded protein in vivo. In addition to traditional excipients such as any and all solvents, dispersion media, diluents, or other liquid vehicles, dispersion or suspension aids, surface active agents, isotonic agents, thickening or emulsifying agents, preservatives, excipients of the present invention can include, without limitation, lipidoids, liposomes, lipid nanoparticles, polymers, lipoplexes, core-shell nanoparticles, peptides, proteins, carbohydrates, cells transfected with polynucleotides (e.g., for transplantation into a subject), hyaluronidase, nanoparticle mimics and combinations thereof. Accordingly, the formulations of the invention can include one or more excipients, each in an amount that together increases the stability of the polynucleotide, increases cell transfection by the polynucleotide, increases the expression of polynucleotides encoded protein, and/or alters the release profile of polynucleotide encoded proteins. Further, the polynucleotides of the present invention may be formulated using self-assembled nucleic acid nanoparticles.

Formulations of the pharmaceutical compositions described herein may be prepared by any method known or hereafter developed in the art of pharmacology. In general, such preparatory methods include the step of associating the active ingredient with an excipient and/or one or more other accessory ingredients.

A pharmaceutical composition in accordance with the present disclosure may be prepared, packaged, and/or sold in bulk, as a single unit dose, and/or as a plurality of single unit doses. As used herein, a “unit dose” refers to a discrete amount of the pharmaceutical composition including a predetermined amount of the active ingredient. The amount of the active ingredient is generally equal to the dosage of the active ingredient which would be administered to a subject and/or a convenient fraction of such a dosage such as, for example, one-half or one-third of such a dosage.

Relative amounts of the active ingredient, the pharmaceutically acceptable excipient, and/or any additional ingredients in a pharmaceutical composition in accordance with the present disclosure may vary, depending upon the identity, size, and/or condition of the subject being treated and further depending upon the route by which the composition is to be administered. For example, the composition may include between 0.1% and 99% (w/w) of the active ingredient. By way of example, the composition may include between 0.1% and 100%, e.g., between 0.5 and 50%, between 1-30%, between 5-80%, at least 80% (w/w) active ingredient.

In some embodiments, the formulations described herein may contain at least one nucleic acid. As a non-limiting example, the formulations may contain 1, 2, 3, 4, 5 or more than 5 nucleic acids described herein.

In one embodiment, the formulation may contain a nucleic acid encoding proteins selected from categories such as, but not limited to, human proteins, veterinary proteins, bacterial proteins, biological proteins, antibodies, immunogenic proteins, therapeutic peptides and proteins, secreted proteins, plasma membrane proteins, cytoplasmic and cytoskeletal proteins, intracellular membrane bound proteins, nuclear proteins, proteins associated with human disease and/or proteins associated with non-human diseases. In one embodiment, the formulation contains at least three nucleic acids encoding proteins. In one embodiment, the formulation contains at least five nucleic acids encoding proteins.

Pharmaceutical formulations may additionally include a pharmaceutically acceptable excipient, which, as used herein, includes, but is not limited to, any and all solvents, dispersion media, diluents, or other liquid vehicles, dispersion or suspension aids, surface active agents, isotonic agents, thickening or emulsifying agents, preservatives, and the like, as suited to the particular dosage form desired. Various excipients for formulating pharmaceutical compositions and techniques for preparing the composition are known in the art (see Remington: The Science and Practice of Pharmacy, 22nd Edition, A. R. Gennaro, Lippincott, Williams & Wilkins, Baltimore, Md., 2012). The use of a conventional excipient medium may be contemplated within the scope of the present disclosure, except insofar as any conventional excipient medium may be incompatible with a substance or its derivatives, such as by producing any undesirable biological effect or otherwise interacting in a deleterious manner with any other component(s) of the pharmaceutical composition.

Pharmaceutically acceptable excipients used in the manufacture of pharmaceutical compositions include, but are not limited to, inert diluents, surface active agents and/or emulsifiers, preservatives, buffering agents, lubricating agents, and/or oils. Such excipients may optionally be included in the pharmaceutical formulations of the invention.

In one embodiment, the polynucleotides of the present invention can be formulated for controlled release and/or targeted delivery. As used herein, “controlled release” refers to a pharmaceutical composition or compound release profile that conforms to a particular pattern of release to effect a therapeutic outcome. In one embodiment, the polynucleotides may be encapsulated into a delivery agent described herein and/or known in the art for controlled release and/or targeted delivery. In one embodiment, the controlled release formulation may include, but is not limited to, tri-block co-polymers. As a non-limiting example, the formulation may include two different types of tri-block co-polymers (International Patent Publication Nos. WO2012131104 and WO2012131106).

In one embodiment, the therapeutic nanoparticle may be formulated for sustained release. As used herein, “sustained release” refers to a pharmaceutical composition or compound that conforms to a release rate over a specific period of time. The period of time may include, but is not limited to, hours, days, weeks, months and years. As a non-limiting example, the sustained release nanoparticle may include a polymer and a therapeutic agent such as, but not limited to, the nucleic acids of the present invention (see International Pub No. WO2010075072 and US Pub No. US20100216804, US20110217377, US20120201859, US20130243848 and US20130243827). In another non-limiting example, the sustained release formulation may include agents which permit persistent bioavailability such as, but not limited to, crystals, macromolecular gels and/or particulate suspensions (see US Patent Publication No US20130150295). In another non-limiting example, the polynucleotides may be formulated in a sustained release formulation as described in International Patent Publication No. WO2014081849. In yet another non-limiting example, the polynucleotides may be delivered in a sustained release formulation according to the methods of International Patent Publication No. WO2014081849.

In another embodiment, the lipid nanoparticle may be encapsulated into any polymer known in the art which may form a gel when injected into a subject. As another non-limiting example, the lipid nanoparticle may be encapsulated into a polymer matrix which may be biodegradable.

In one embodiment, the polynucleotides may be delivered in therapeutic nanoparticles for parenteral administration.

In one embodiment, the polynucleotides of the present invention may be formulated in the immunostimulatory protamine-RNA nanoparticle composition described in or made by the methods described in European Patent Publication No. EP2306993B1.

### EXAMPLES

### Example 1. Injector Design

An exemplary injector for use as part of any of the methods or apparatus described herein is shown in FIG. 1. A stepper motor or a servo motor, located at the top of the injector, powers a plunger within a syringe barrel. The plunger is connected to the motor by a lead screw. A pipette tip adaptor is attached to the bottom of the syringe to receive liquid from the inside of the syringe barrel, which can be ejected by downward motion of the plunger. The pipette tip adaptor holds a disposable pipette tip in place, forming a hermetic seal.

### Example 2. Nanoparticle Synthesis

An exemplary protocol executable by an apparatus of the present invention is provided herein. An injector (e.g., as described in Example 1) initiates a nanoparticle synthesis protocol by attaching a clean pipette tip from a tip holder. The injector is then moved laterally to a position over a reservoir containing a lower alkanol solution. In this case, the reservoir containing the lower alkanol solution is a 10 ml microcentrifuge tube held in place by a holder of the apparatus. The injector is then moved downwards to bring the tip in contact with the lower alkanol solution, and a servo pump provides negative pressure to the tip, bringing 600 μl of the lower alkanol solution into the tip. The injector is then brought vertically out of the lower alkanol solution reservoir and moved laterally to a position over an aqueous solution reservoir containing 900 μl of aqueous solution containing 100 μg mRNA. In this case, the aqueous solution reservoir is a 10 ml polystyrene tube secured in a 24-tube rack positioned within the apparatus. The injector is then lowered to bring the tip into contact with the aqueous solution, and the servo pump pressurizes the tip to force a stream of the 600 μl of lower alkanol solution into the aqueous solution at a rate of 2,600 μl/second for about 0.23 seconds. After injection, the injector is brought out of the reservoir and moved to a tip stripper, which removes the used pipette tips. This method can be scaled linearly. The process is repeated as required.

### Other Embodiments

It is to be understood that while the present disclosure has been described in conjunction with the detailed description thereof, the foregoing description is intended to illustrate and not limit the scope of the present disclosure, which is defined by the scope of the appended claims. Other aspects, advantages, and alterations are within the scope of the following claims.

What is claimed is:

1. A method for producing lipid nanoparticles, the method comprising:
a. providing an aqueous solution;
b. providing a lower alkanol solution comprising lipids; and
c. injecting the lower alkanol solution to the aqueous solution at an automated rate to produce the lipid nanoparticles.

2. The method of claim 1, further comprising automatically repeating steps (a)-(c) one or more times.

3. The method of claim 1 or 2, wherein the injecting is powered by a servo pump.

4. The method of any one of claims 1-3, wherein the lipid nanoparticles have a mean diameter between 80 and 100 nanometers and a polydispersity index of 0.25 or less.

5. The method of any one of claims 1-4, wherein the aqueous solution comprises a nucleic acid.

6. The method of claim 5, wherein the nucleic acid is at a concentration between 50 μg per ml of the aqueous solution and 200 μg per ml of the aqueous solution.

7. The method of claim 6, wherein the nucleic acid is at a concentration of about 111 μg per ml of the aqueous solution.

8. The method of any one of claims 5-7, wherein all or a portion of the nucleic acid is encapsulated in the lipid nanoparticles.

9. The method of claim 8, wherein the process yields a nucleic acid encapsulation efficiency of at least 94%.

10. The method of any one of claims 5-9, wherein the nucleic acid is mRNA.

11. The method of any one of claims 1-10, wherein the lower alkanol solution provides between 25% and 50% of the total volume after injecting.

12. The method of claim 11, wherein the lower alkanol solution provides about 40% of the total volume.

13. The method of any one of claims 1-12, wherein the injecting is at a rate from 1,000 to 5,000 microliters per second (μl/s).

14. The method of claim 13, wherein the injecting is at a rate from 2,500 to 3,000 μl/s.

15. The method of claim 14, wherein the rate is 2,600 μl/s.

16. The method of any one of claims 1-15, wherein the aqueous solution further comprises a citrate buffer.

17. The method of any one of claims 1-16, wherein the aqueous solution has a pH from 6.0 to 8.0.

18. The method of any one of claim 17, wherein the aqueous solution has a pH of about 7.4.

19. The method of any one of claims 1-18, wherein the lower alkanol solution comprises heptatriaconta-6,9,28,31-tetraen-19-yl-4-(dimethylamino)butanoate (DLin-MC3-DMA), phosphatidylcholine (1,2-distearoyl-sn-glycero-3-phosphocholine (DSPC), cholesterol, and polyethylene glycol-dimyristolglycerol (PEG-DMG).

20. The method of claim 19, wherein the ratio of DLin-MC3-DMA:DSPC:Cholesterol:PEG-DMG is 50:10:38.5:1.5.

21. The method of any one of claims 1-20, further comprising purifying or concentrating the dispersion of lipid nanoparticles by tangential flow filtration, dialysis, or desalting.

22. The method of any one of claims 1-21, further comprising sterilizing the dispersion of lipid nanoparticles by microfiltration.

23. A lipid nanoparticle produced by injecting a lower alkanol solution into an aqueous solution comprising a lipid, wherein the injecting is automated at a rate of 2,600 μl/s.

24. An mRNA-encapsulated lipid nanoparticle having a mean diameter between 80 and 100 nanometers and a polydispersity index of 0.25 or less.

25. An apparatus for producing lipid nanoparticles, the apparatus comprising:
a. an injector configured to transfer a lower alkanol solution from a first reservoir to a second reservoir configured to hold an aqueous solution; and
b. a servo pump configured to operate the injector at a rate from 1,000 to 5,000 μl/s.

26. The apparatus of claim 25, wherein the servo pump is configured to operate the injector at a rate between 1,000 μl/s and 5,000 μl/s.

27. The apparatus of claim 26, wherein the servo pump is configured to operate the injector at a rate of about 2,600 μl/s.

28. The apparatus of claim 25 or 27, wherein the injector is configured to move in three dimensions relative to the second reservoir.

29. The apparatus of any one of claims 25-28, wherein the injector is configured to move in three dimensions relative to the first reservoir and the second reservoir.

30. A pharmaceutical composition comprising the lipid nanoparticles of any one of claims 1-24 and a pharmaceutically acceptable carrier.
